(19) United States
(12) Patent Application Publication
(10) Pub. No.: US 2003/0125389 A1
(43) Pub. Date: Jul. 3, 2003

(54) Beta-amino acid derivatives useful for the treatment of bacterial infections

(76) Inventors: Bore G. Raju, Fremont, CA (US); Sampathkumar Anandan, Fremont, CA (US); Joaquim Trias, Millbrae, CA (US); Prudencio S. Herradura, Berkeley, CA (US); Kathleen H. Mortell, Chicago, IL (US); Dinesh V. Patel, Fremont, CA (US)

Correspondence Address: Gerald F. Swiss, BURNS, DOANE, SWECKER & MATHIS, L.L.P., P.O. Box 1404, Alexandria, VA, 22313-1404, US

(21) Appl. No.: 10/202,630

(22) Filed: Jul. 25, 2002

Related U.S. Application Data
(60) Provisional application No. 60/307,875, filed on Jul. 25, 2001.

(51) Int. Cl. A61K 31/198; A61K 31/44; A61K 31/40; C07D 213/56; C07C 243/10; C07C 239/14

(52) U.S. Cl. 514/563; 514/357; 514/408; 562/560; 546/336; 548/567

(57) ABSTRACT

The invention provides compounds that are useful for the treatment of bacterial infections in mammals. More specifically, it is directed to beta-amino acid derivatives or pharmaceutically acceptable salts, prodrugs, or isomers of

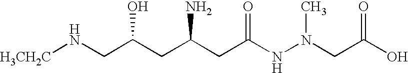

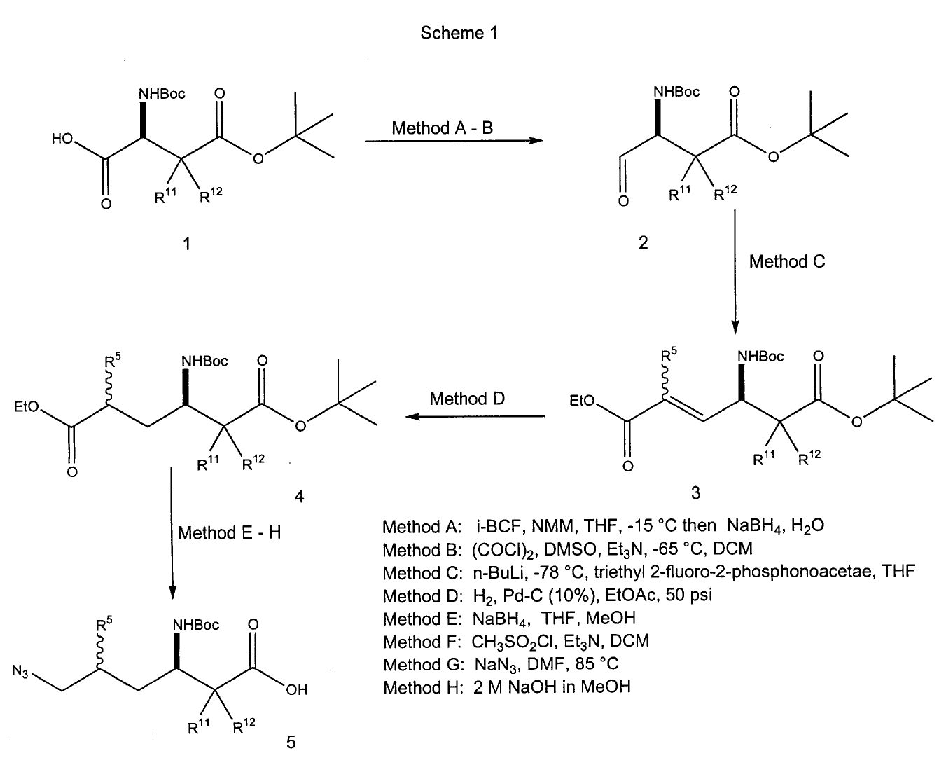

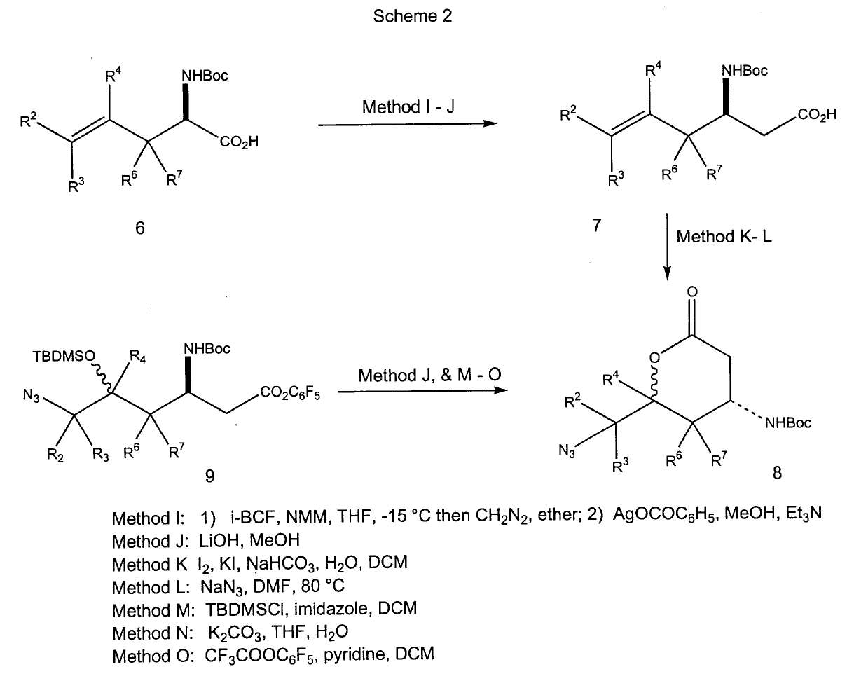

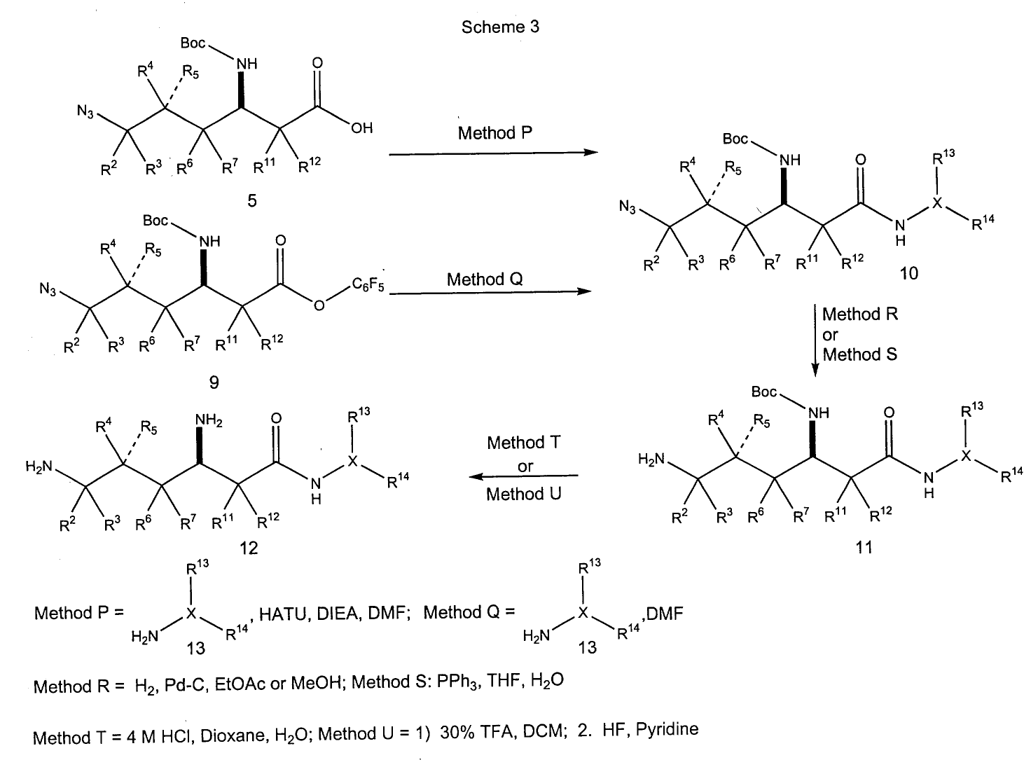

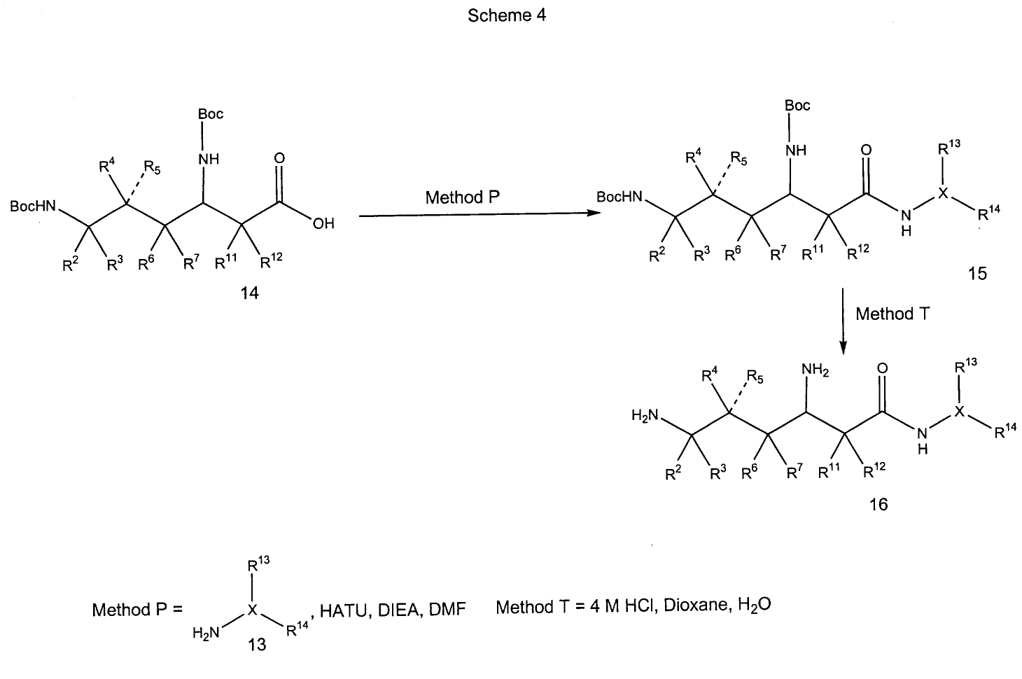

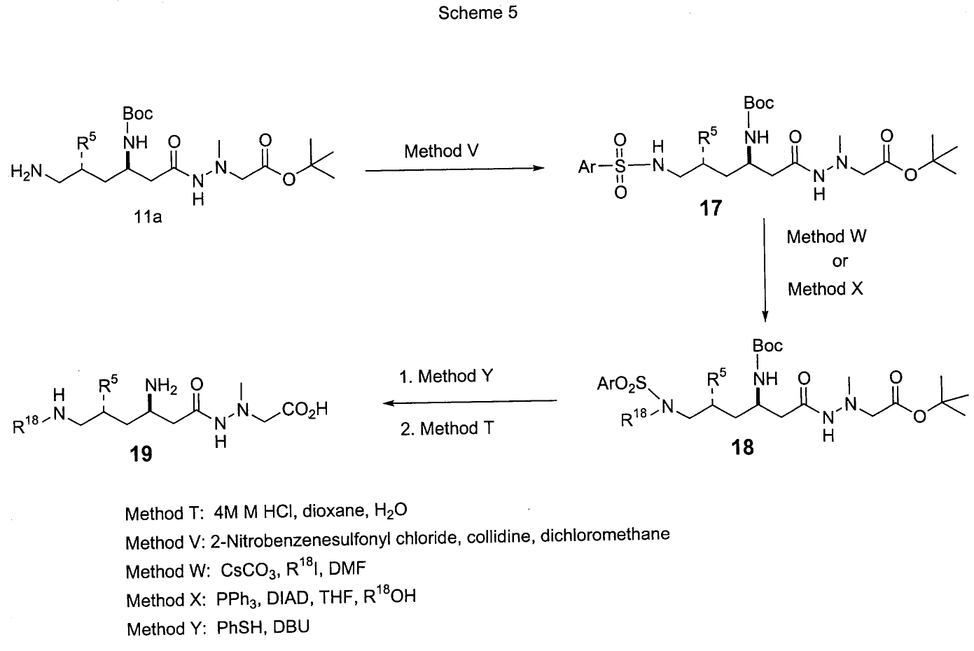

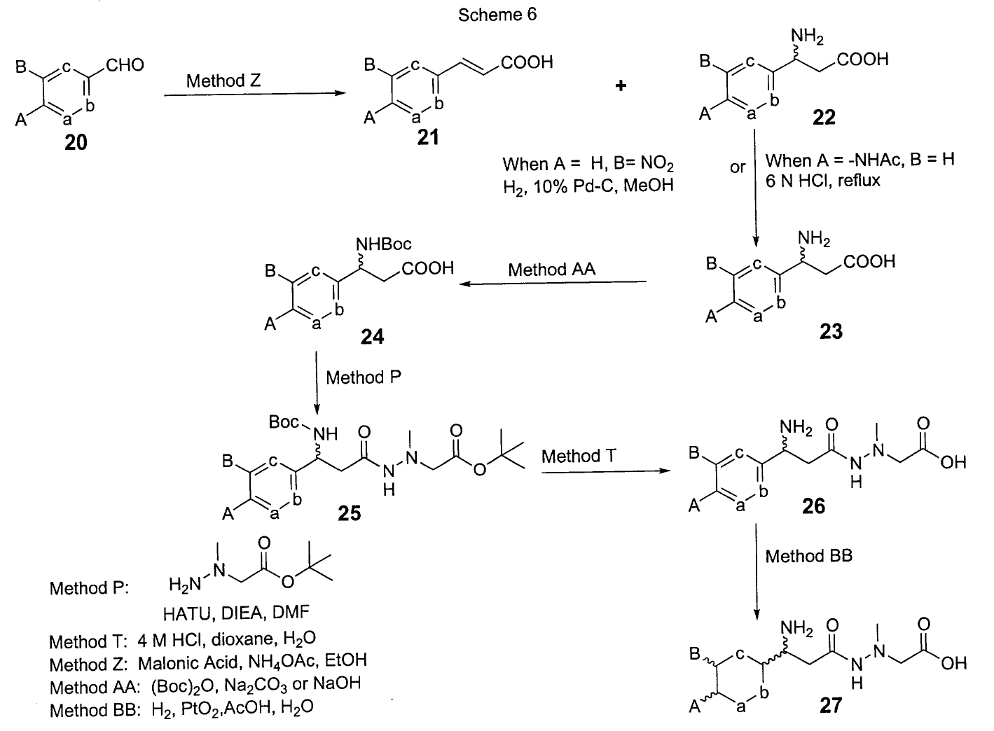

those compounds that are useful for the treatment of such infections.

### CROSS-REFERENCE TO RELATED APPLICATIONS

This application claims the benefit of U.S. Provisional Application No. 60/307,875, filed Jul. 25, 2001, which is herein incorporated by reference in its entirety.

### BACKGROUND OF THE INVENTION

1. Field of the Invention

The present invention is generally related to compounds that are useful for the treatment of bacterial infections in mammals. More particularly, it is directed to beta-amino acid derivatives or pharmaceutically acceptable salts, prodrugs, or isomers of those compounds that are useful for the treatment of such infections.

### REFERENCES

The following publications, patents and patent applications are cited in this application as superscript numbers:

1 M. Hamada et al., “A New Antibiotic, Negamycin,” J. Antibiotics, 23(3):170-71 (1970).

2 Umezawa et al., “Negamycin,” U.S. Pat. No. 3,679,742, issued Jul. 25, 1972.

3 S. Kondo, et al., “Negamycin, a Novel Hydrazide Antibiotic,” J. Am. Chem. Soc., 93(23), 6305-6 (1971).

4 S. Shibahara et al., “The Total Synthesis of Negamycin and the Antipode,” J. Am. Chem. Soc. 94:4353-54 (1972).

5 Umezawa et al., “Antibiotic, Negamycin, and Processes for the Preparation Thereof,” U.S. Pat. No. 3,743,580 issued Jul. 3, 1973

6 W. Streicher, “Total Synthesis of Rac. Negamycin and of Negamycin Analogs,” J. Antibiotics, 31(7):725-728 (1978).

7 A. Pierdet et al., “Synthese Totale de la (±)-Negamycine,” Tetrahedron, 36:1763-1772 (1980).

8 G. Pasquet et al., “Synthesis of (±)-Negamycin and of (±)-Epinegamycin”, Tetrahedron Letters, 21:931-934 (1980).

9 Y. -F. Wang et al., “Stereocontrolled Synthesis of (+)-Negamycin from an Acyclic Homoallylamine by 1,3-Asymmetric Induction,” J. Am. Chem. Soc., 104:6466-6468 (1982)

10 H. Iida et al., “Enantioelective Total Synthesis of (+)-Negamycin and (−)-Epinegamycin by an Aymmetric 1,3-Dipolar Cycloaddition,” J. Am. Chem. Soc., 108:4647-4648 (1986).

11 S. De Bernardo et al., “Synthesis of (+)-Negamycin from D-Glucose,” Tetrahedron Letters, 29(33):4077-4080 (1988).

12 D. Tanner et al., “Enantioselective Total Synthesis of (+)-Negamycin,” Tetrahedron Letters, 29(19):2373-2376 (1988).

13 D. Tanner, “Total Synthesis of Natural Products: Some General Observations and a Specific Example. An Enantioselective Route to (+)-Negamycin,” Acta Pharm. Nord., 1(3):145-154 (1989).

14 K. Kasahara et al., “Asymmetric Total Synthesis of (+)-Negamycin and (−)-3-Epinegamycin via Enantioselective 1,3-Dipolar Cycloaddition,” J. Org. Chem, 54:2225-33 (1989).

15 C. D. Maycock et al., “An Application of Quinic Acid to the Synthesis of Linear Homochiral Molecules: A Synthesis of (+)-Negamycin,” Tetrahedron Letters, 33(32):4633-4636 (1992).

16 J. J. Masters and L. S. Hegedus, “Palladium(II)-Assisted Difunctionalization of Monoolefins: Total Synthesis of (+)-Negamycin and (−)-5-epi-Negamycin,” J. Org. Chem., 58:4547-4554 (1993).

17 D. Socha et al., “Stereocontrolled Entry to Negamycin from D-Glucose,” Tetrahedron Letters, 36(1): 135-138 (1995).

18 S. G. Davies and O. Ichihara, “Asymmetric Synthesis of (+)-Negamycin,” Tetrahedron: Asymmetry, 7(7):1919-1922 (1996).

19 M. Shimizu et al., “Stereocontrol in the Addition of Allyl Metal Reagents to an Optically Active Imine Derived from Malic Acid, Leading to a Formal Synthesis of (+)-Negamycin,” Chemistry Letters, 467-8 (1998).

20 S. Kondo et al., “Synthesis and Properties of Negamycin Analogs Modified the δ-hydroxy-β-lysine moiety,” J. Antibiotics, 29(2):208-211 (1976).

21 W. V. Curran, “D-3,6-Diaminohexanoic acid 2-(carboxymethyl)-2-methylhydrazide, Process of Preparing and Intermediates of Said Process,” U.S. Pat. No. 3,962,317 issued Jun. 8, 1976.

22 W. V. Curran and J. H. Boothe, “The Synthesis of Deoxynegamycin and Some Related Compounds,” J. Antibiotics, 31(9):914-918(1978).

23 S. Mizuno et al., “Mechanism of Action of Negamycin in Escherichia Coli K12; I. Inhibition of Initiation of Protein Synthesis,” J. Antibiotics, 23(12):581-588 (1970).

24 S. Mizuno et al., “Mechanism of Action of Negamycin in Escherichia Coli K12; II. Miscoding Activity in Polypeptide Synthesis Directed by Synthetic Polynucleotide,” J. Antibiotics, 23(12):589-594 (1970).

25 “Negamycin Derivatives and Synthesis Thereof,” GB 1,553,416, published Sep. 26, 1979.

26 Umezawa et al., “δ-Substituted Negamycin Derivatives and Syntheses,” U.S. Pat. No. 4,065,495 issued Dec. 27, 1977.

27 S. Kondo et al., “Leucylnegamycin, an antibiotic from negamycin-producing Streptomyces,” J. Antibiotics, 24:732-4 (1971).

28 W. Streicher, et al., “Synthese eines Azaanalogen des Antibiotikums Negamycin,” Chem. Ber., 108:813-9 (1975).

29 Y. Uehara, et al., “Structure-Activity Relationships Among Negamycin Analogs,” J. Antibiotics, 29(9):937-943 (1976).

30 S. Konda et al., “3-Epi-Deoxynegamycin and Leucyl-3-Epi-Deoxynegamycin Produced by Streptomyces,” J. Antibiotics, 30(12):1137-9 (1977).

31 N. Katayama, et al., “Sperabillins, New Antibacterial Antibiotics with Potent In Vivo Activity,” J. Antibiotics, 45(1):10-19 (1992).

32 T. Hida, et al., “Structures of New Pseudo-Peptide Antibiotics, Sperabillins,” Bull. Chem. Soc. Jpn., 66:863-869 (1993).

33 S. Hashiguchi, et al., “Steroselective Synthesis of Sperabillins and Related Compounds,” J. Chem. Soc. Perkin Trans., 1:2435-44 (1991).

34 T. Hida, et al., “Synthesis and Antimicrobial Activity of Sperabillin Derivatives,” J. Antibiotics, 46(5):803-812 (1993).

35 T. Hida, et al., “Chemistry and Anti-tumor Activity of Sperabillin Polymers,” Chem. Pharm. Bull., 41(5):889-93 (1993).

36 M. Takizawa, et al., “Augmentation of Host Defense Mechanisms Against Tumor by Sperabillin Polymers, New Basic Peptidyl Biopolymers, in Mice,” Int. J. Immunopharmac., 16(1):67-74 (1994).

All of the above publications, patents and patent applications are herein incorporated by reference in their entirety to the same extent as if each individual publication, patent or patent application was specifically and individually to be incorporated by reference in its entirety.

2. State of the Art

Negamycin is an antibiotic, isolated from the culture filtrate of three strains related to Streptomyces purpeofuscus, and was first reported by M. Hamada et al.¹ Its chemical structure is shown below:

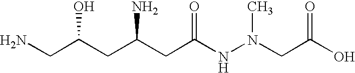

[2-{(3R, 5R)-3,6-diamino-5-hydroxyhexanoyl}-1-methylhydrazino]acetic acid

Negamycin may be produced by cultivating a strain of S. purpeofurcus in a nutrient medium under aerobic conditions, according to the protocol set forth in U.S. Pat. No. 3,679,742.² Alternatively, negamycin may also be synthesized, for example, by the reaction of (R,R)-δ-hydroxy-β-lysine and 1-methylhydrazinoacetic acid, as described by S. Kondo, et al.,³ and S. Shibahara, et al.⁴ A variety of other synthetic methods for making negamycin are also described in the art,⁵⁻⁸ including a variety of enantioselective syntheses.⁹⁻¹⁹

Deoxynegamycin is the deoxy analog of negamycin, and has the following structure:

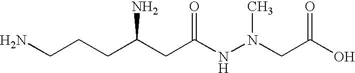

D-3,6-diaminiohexanoic acid-2-(carboxymethyl)-2-methylhyrazide

The synthesis of deoxynegamycin, using negamycin as starting material, was first reported by S. Kondo et al.²⁰ Various other methods for the synthesis of deoxynegamycin have since been reported.²¹⁻²²

Both negamycin and deoxynegamycin display antibacterial activity. Negamycin displays low toxicity and displays strong inhibitory activity against Gram negative and Gram positive bacteria. Negamycin has been shown to inhibit initiation of protein synthesis,²³ and to induce misreading of the genetic codes of synthetic mRNAs.24 Negamycin has been reported as effective for inhibiting the following organisms:

Staphylococcus aureus^{1,4,7,20,22}

Escherichia coli^{1,4,7,20,22}

Shigella sonnei⁴

Shigella flexneri^{1,22}

Salmonella typhosa^{1,7}

Salmonella typhi^{4,20}

Salmonella typhimurium²²

Salmonella enteriditis²²

Enterobacter sp.²²

Klebsiella pneumoniae^{7,20,22}

Serratia marcescens^{1,20,22}

Herellea vaginicola²²

Proteus vulgaris^{1,20,22}

Proteus rettgeri^{1,26}

Pseudomonas aeruginosa^{1,4,7,20,22}

Pseudomonas fluorescens²⁰

Mycobacterium smegmatis^{1,20}

Deoxynegamycin is reported to be useful as an antimicrobial agent, with broad-spectrum antibacterial and antifungal activity in vitro against a variety of standard laboratory microorganisms. Deoxynegamycin is reported as effective against the following organisms:

Staphylococcus aureus^{20,21,22,26}

Escherichia coli^{20,21,22,26}

Shigella flexneri^{21,22}

Salmonella typhi^{20,26}

Salmonella typhimurium^{21,22}

Salmonella enteriditis^{21,22}

Klebsiella pneumoniae^{20,21,22,26}

Enterobacter sp^{21,22}

Enterobacter aerogenes²¹

Serratia marcescens^{20,21,22,26}

Herellea vaginicola²¹

Proteus mirabilis^{21,22}

Proteus vulgaris^{20,21,22,26}

Proteus rettgeri^{20,26}

Pseudomonas aeruginosa^{20,21,26}

Pseudomonas fluorescens^{20,26}

Mycobacterium smegmatis^{20,21,26}

Candida albicans²¹

Cryptococcus neoformans²¹

Trichophyton tonsurans²¹

Trichophyton mentagraphytes²¹

Derivatives and analogs of both negamycin and deoxynegamycin have been pursued.²⁹ For instance, the antipode of negamycin,⁴ as well as diastereomeric analogs of negamycin⁶ have been reported. In addition, leucylnegamycin,²⁷ O-methylnegamycin,²⁰ and di-N-benzyloxycarbonylnegamycin methyl ester²⁰ have been synthesized. Various aza-analogs of negamycin,²⁸ C-6 methyl derivatives of negamycin,³³ and analogs where the hydrazinoacetic acid moiety of negamycin was modified⁶ are also reported in the art.

Analogs of deoxynegamycin are also known in the art. The discovery of 3-epi-deoxynegamycin and leucyl-3-epi-deoxynegamycin produced by Streptomyces was reported by Kondo.³⁰ The synthesis of 3-epi-deoxynegamycin²⁰ has been reported, as well as the synthesis of a variety of derivatives where the N-methylhydrazinoacetic acid moiety is replaced.²²

The sperabillin compounds are produced by the bacterium Pseudomonas fluorescence YK-437,^{31,32} and are reported to have antibacterial activity.³³ Their chemical structures are shown below:^{32,33}

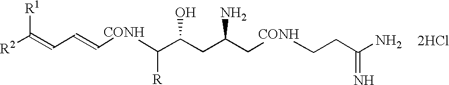

Sperabillin A-D

A: R¹=Me, R²=R=H

B: R¹=R=Me, R²=H

C: R¹=R=H, R²=Me

D: R¹=H, R²=R=Me

As shown above, the sperabillin compounds have a 3,6-diamino-5-hydroxyhexanoic acid moiety, also found in negamycin and deoxynegamycin.³³ Various derivatives of the sperabillin compounds have been reported,^{33,34} as well as polymers based on sperabillin subunits.^{35,36}

However, despite these developments, as reported by several research groups, the derivatives and analogs known in the art have not produced compounds as active as negamycin.^{4,27-29,33} Thus, there remains a need for compounds that can be used to treat bacterial infections. This invention answers this need.

### SUMMARY OF THE INVENTION

The invention relates generally to compounds that are useful for the treatment of bacterial infections in mammals. Specifically, the invention is directed to beta-amino acid derivatives, or pharmaceutically acceptable salts, prodrugs or isomers thereof, that are useful in the treatment of bacterial infections.

Accordingly, in one of its compound aspects, this invention is directed to a compound of formula (I):

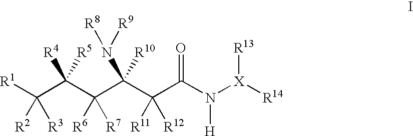

wherein,

R¹ is —NHR¹⁸, wherein R¹⁸ is H, CH₃ or CH₂CH₃;

R² is H, alkyl or R² and either R⁴ or R⁵ together with the atoms to which they are attached form a cyclic alkyl group, or R² and R⁶ together with the atoms to which they are attached form a cyclic alkyl group, an aryl group or a heteroaryl group, or R² and R⁸ together with the atoms to which they are attached form a cyclic heteroalkyl group;

R³ is H, alkyl or R³ and R¹⁰ together with the atoms to which they are attached form a cyclic alkyl group;

R⁴ is H, alkyl or R⁴ and R⁶ together with the atoms to which they are attached form a cyclic alkyl group, or R⁴ and R⁸ together with the atoms to which they are attached form a cyclic heteroalkyl group, or R⁴ and R¹⁰ together with the atoms to which they are attached form a cyclic alkyl group;

R⁵ is H, F, Cl , OR¹⁶, SR¹⁶ or S(O)ₙR¹⁷, or R⁵ and R⁶ together with the atoms to which they are attached form a cyclic alkyl group, or R⁵ and R⁸ together with the atoms to which they are attached form a cyclic heteroalkyl group;

R⁶ is H, alkyl or R⁶ and R² together with the atoms to which they are attached form a cyclic alkyl group, an aryl group or a heteroaryl group, or R⁶ and R⁴ together with the atoms to which they are attached form a cyclic alkyl group, or R⁶ and R⁵ together with the atoms to which they are attached form a cyclic alkyl group;

R⁷ is H, alkyl or R⁷ and R¹⁰ together with the atoms to which they are attached form a cyclic alkyl group;

R⁸ is H, alkyl, or R⁸ and R² together with the atoms to which they are attached form a cyclic heteroalkyl group, or R⁸ and R⁴ together with the atoms to which they are attached form a cyclic heteroalkyl group, or R⁸ and R⁵ together with the atoms to which they are attached form a cyclic heteroalkyl group, or R⁸ and R⁶ together with the atoms to which they are attached form a cyclic heteroalkyl group, or R⁸ and R¹¹ together with the atoms to which they are attached form a cyclic heteroalkyl group;

R⁹ is H or alkyl;

R¹⁰ is H or alkyl, or R¹⁰ and R³ together with the atoms to which they are attached form a cyclic alkyl group, or R¹⁰ and R⁴ together with the atoms to which they are attached form a cyclic alkyl group, or R¹⁰ and R⁷ together with the atoms to which they are attached form a cyclic alkyl group, or R¹⁰ and R¹¹ together with the atoms to which they are attached form a cyclic alkyl group;

R¹¹ is H, alkyl, OH, SH or F, or R¹¹ and R⁸ together with the atoms to which they are attached form a cyclic heteroalkyl group, or R¹¹ and R¹⁰ together with the atoms to which they are attached form a cyclic alkyl group;

R¹² is H or alkyl;

R¹³ is H or alkyl when X is N;

R¹³ is not a substituent when X is O;

R¹⁴ is —CHR¹⁵—CO₂H, —C₆H₄—CO₂H, —C₄H₃N—CO₂H, —C₄H₂S—CO₂H, C₄H₂O—CO₂H, —CH R¹⁵—S(O)₂OH, —CH R¹⁵—S(O)₂—NH₂ or —CH R¹⁵—P(O)(CH₃)OH;

R¹⁵ is H or alkyl;

R¹⁶ is H or alkyl;

R¹⁷ is alkyl or aryl;

R¹⁸ is H, CH₃ or CH₂CH₃; and,

n is 1 or 2;

with the proviso that the compound of formula (I) is neither negamycin nor deoxynegamycin.

In one preferred compound embodiment, R¹⁴ is —CHR¹⁵—CO₂H.

In another preferred compound embodiment, R⁵ is OH.

In another preferred compound embodiment, R⁵ is F.

In another preferred compound embodiment, R² and R⁶ together with the atoms to which they are attached form a cyclic alkyl group, an aryl group or a heteroaryl group.

In another preferred compound embodiment, R² and R⁸ together with the atoms to which they are attached form a cyclic alkyl group.

In another preferred compound embodiment, either R⁴ or R⁵ and R⁶ together with the atoms to which they are attached form a cyclic alkyl group.

In another preferred compound embodiment, either R⁴ or R⁵ and R⁸ together with the atoms to which they are attached form a cyclic heteroalkyl group.

In another preferred compound embodiment, R¹ is —NHR¹⁸, X is N, and R², R³, R⁴, R⁶, R⁷, R⁸, R⁹, R¹⁰, R¹¹ and R¹² are H.

In another preferred compound embodiment, R² and R⁶ together with the atoms to which they are attached form a cyclic alkyl group, wherein the cyclic alkyl group is selected from the group consisting of the following cyclic alkyl groups: cyclopropyl, cyclobutyl, cyclopentyl and cyclohexyl.

In another preferred compound embodiment, R¹ is —NHR¹⁸, X is N, substituents R², R³, R⁴, R⁶, R⁷, R⁸, R⁹, R¹⁰, R¹¹ and R¹² are H, R¹³ is methyl and R¹⁴ is —CHR¹⁵—CO₂H.

In another preferred compound embodiment, R² and R⁶ together with the atoms to which they are attached form a cyclopropyl group.

In another preferred compound embodiment, R¹ is —NHR¹⁸, X is N, substituents R², R³, R⁴, R⁶, R⁷, R⁸, R⁹, R¹⁰, R¹¹ and R¹² are H, R¹³ is methyl, R¹⁴ is —CHR¹⁵—CO₂H and R¹⁵ is H.

In another preferred compound embodiment, the compound is selected from a group consisting of the following compounds:

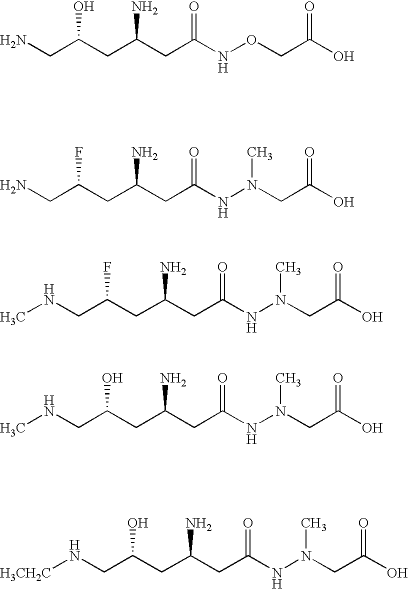

In one of its method aspects, this invention is directed to a method of treating a bacterial infection in a mammal. The method comprises administering a compound of formula (I), as described above, to the mammal.

In one preferred method embodiment, a compound of formula (I) is administered in conjunction with another antibiotic, preferably a gram positive agent.

In one of its composition aspects, this invention is directed to a composition comprising a pharmaceutically acceptable carrier and a therapeutically effective amount of a compound of formula (I), as described above.

### BRIEF DESCRIPTION OF THE DRAWINGS

Schemes 1-6 show general synthetic routes to beta-amino acid derivatives of the present invention.

### DETAILED DESCRIPTION OF THE INVENTION

As above, this invention relates to the treatment of bacterial infections in mammals. More particularly, the invention is directed to beta-amino acid derivatives, or pharmaceutically acceptable salts, prodrugs or isomers thereof, that are useful in the treatment of bacterial infections. However, prior to describing the invention in further detail, the following terms will first be defined.

A. Definitions

“Bacterial Infection” refers to an infection resulting from the invasion of the body by bacteria. The infection may or may not be clinically apparent. “Bacteria” refers to unicellular microorganisms of the class Schizomycetes, as well as all prokaryotic organisms that are not blue-green algae. Bacteria may have a variety of shapes, for example, spheric (cocci), rod-shaped (bacilli), spiral (spirochetes), or comma-shaped (vibrio). The invasion of the body by bacteria may cause disease in a variety of ways, including but not limited to: local cellular injury, competitive metabolism, secretion of a toxin, or an antigen-antibody reaction in the host.

“Negamycin” refers to [2-{(3R, 5R)-3,6-diamino-5-hydroxyhexanoyl}-1-methylhydrazino]acetic acid, and has the following chemical formula:

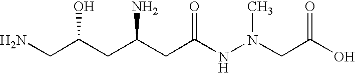

“Deoxynegamycin” refers to D-3,6-diaminiohexanoic acid-2-(carboxymethyl)-2-methylhyrazide, and has the following chemical formula:

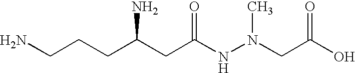

The term “alkyl” refers to saturated aliphatic groups including straight-chain, branched-chain, cyclic groups, and combinations thereof, having the number of carbon atoms specified, or if no number is specified, having 1 to 12 carbon atoms. Examples of alkyl groups include, but are not limited to, groups such as methyl, ethyl, n-propyl, isopropyl, n-butyl, isobutyl, sec-butyl, t-butyl, n-pentyl, neopentyl, cyclopropyl, cyclobutyl, cyclopentyl, cyclohexyl, cyclobutylmethyl, cyclobutylethyl, cyclopentylmethyl, cyclopentylethyl, and adamantyl. Cyclic alkyl groups can consist of one ring, including, but not limited to, groups such as cycloheptyl, or multiple fused rings, including, but not limited to, groups such as adamantyl or norbornyl.

The term “alkylene” means a saturated divalent aliphatic groups including straight-chain, branched-chain, cyclic groups, and combinations thereof, having the number of carbon atoms specified, or if no number is specified, having 1 to 12 carbon atoms, e.g., methylene, ethylene, 2,2-dimethylethylene, propylene, 2-methyl-propylene, butylene, pentylene, cyclopentylmethylene, and the like.

The term “substituted alkyl” means an alkyl group as defined above that is substituted with one or more substituents, preferably one to three substituents selected from the group consisting of halogen (fluoro, chloro, bromo, and iodo, preferably fluoro, chloro, or bromo), alkoxy, acyloxy, amino, mono or dialkylamino, hydroxyl, mercapto, carboxy, benzyloxy, phenyl, benzyl, cyano, nitro, thioalkoxy, carboxaldehyde, carboalkoxy and carboxamide, or a functionality that can be suitably blocked, if necessary for purposes of the invention, with a protecting group. The phenyl group may optionally be substituted with one to three substituents selected from the group consisting of halogen (fluoro, chloro, bromo, and iodo, preferably fluoro, chloro, or bromo), alkoxy, acyloxy, amino, mono or dialkylamino, hydroxyl, mercapto, carboxy, benzyloxy, benzyl, cyano, nitro, thioalkoxy, carboxaldehyde, carboalkoxy and carboxamide. Examples of substituted alkyl groups include, but are not limited to, —CF3, —CF2—CF3, hydroxymethyl, 1- or 2-hydroxyethyl, methoxymethyl, 1- or 2-ethoxyethyl, carboxymethyl, 1- or 2-carboxyethyl, methoxycarbonylmethyl, 1- or 2-methoxycarbonyl ethyl, benzyl, and the like.

The term “substituted alkylene” means an alkylene group as defined above that is substituted with one or more substituents, preferably one to three substituents, selected from the group consisting of halogen (fluoro, chloro, bromo, and iodo, preferably fluoro, chloro, or bromo), alkoxy, acyloxy, amino, mono or dialkylamino, hydroxyl, mercapto, carboxy, benzyloxy, phenyl, benzyl, cyano, nitro, thioalkoxy, carboxaldehyde, carboalkoxy and carboxamide, or a functionality that can be suitably blocked, if necessary for purposes of the invention, with a protecting group. The phenyl group may optionally be substituted with one to three substituents selected from the group consisting of halogen (fluoro, chloro, bromo, and iodo, preferably fluoro, chloro, or bromo), alkoxy, acyloxy, amino, mono or dialkylamino, hydroxyl, mercapto, carboxy, benzyloxy, benzyl, cyano, nitro, thioalkoxy, carboxaldehyde, carboalkoxy and carboxamide. Examples of substituted alkyl groups include, but are not limited to, —CF2—, —CF2—CF2—, hydroxymethylene, 1- or 2-hydroxyethylene, methoxymethylene, 1- or 2-ethoxyethylene, carboxymethylene, 1- or 2-carboxy-ethylene, and the like.

The term “alkenyl” refers to unsaturated aliphatic groups including straight-chain, branched-chain, cyclic groups, and combinations thereof, having the number of carbon atoms specified, or if no number is specified, having 1 to 12 carbon atoms, which contain at least one double bond (—C═C—). Examples of alkenyl groups include, but are not limited to, allyl vinyl, —CH2—CH═CH—CH3, —CH2—CH2-cyclopentenyl and —CH2—CH2-cyclohexenyl where the ethyl group can be attached to the cyclopentenyl, cyclohexenyl moiety at any available carbon valence.

The term “alkenylene” refers to unsaturated divalent aliphatic groups including straight-chain, branched-chain, cyclic groups, and combinations thereof, having the number of carbon atoms specified, or if no number is specified, having 1 to 12 carbon atoms, which contain at least one double bond (—C═C—). Examples of alkenylene groups include, but are not limited to, —CH═CH—, —CH₂—CH═CH—CH₂—, —CH₂—CH(cyclopentenyl)—and the like.

The term “alkynyl” refers to unsaturated aliphatic groups including straight-chain, branched-chain, cyclic groups, and combinations thereof, having the number of carbon atoms specified, or if no number is specified, having 1 to 12 carbon atoms, which contain at least one triple bond (—CC—). Examples of alkynyl groups include, but are not limited to, acetylene, 2-butynyl, and the like.

The term “alkynylene” refers to unsaturated divalent aliphatic groups including straight-chain, branched-chain, cyclic groups, and combinations thereof, having the number of carbon atoms specified, or if no number is specified, having 1 to 12 carbon atoms, which contain at least one triple bond (—CC—). Examples of alkynylene groups include, but are not limited to, —CC—, —CC—CH₂—, and the like.

The term “substituted alkenyl” or “substituted alkynyl,” refers to the alkenyl and alkynyl groups as defined above that are substituted with one or more substituents, selected from the group consisting of halogen, alkoxy, acyloxy, amino, hydroxyl, mercapto, carboxy, benzyloxy, phenyl, benzyl, cyano, nitro, thioalkoxy, carboxaldehyde, carboalkoxy and carboxamide, or a functionality that can be suitably blocked, if necessary for purposes of the invention, with a protecting group. Examples of substituted alkenyl and alkynyl groups include, but are not limited to, —CH═CF₂, hydroxyethenyl, methoxypropenyl, hydroxypropynyl, and the like.

The term “substituted alkenylene” or “substituted alkynylene,” refers to the alkenylene and alkynylene groups as defined above that are substituted with one or more substituents, selected from the group consisting of halogen, alkoxy, acyloxy, amino, hydroxyl, mercapto, carboxy, benzyloxy, phenyl, benzyl, cyano, nitro, thioalkoxy, carboxaldehyde, carboalkoxy and carboxamide, or a functionality that can be suitably blocked, if necessary for purposes of the invention, with a protecting group.

The term “aryl” or “Ar” refers to an aromatic carbocyclic group of 6 to 14 carbon atoms having a single ring (including, but not limited to, groups such as phenyl) or multiple condensed rings (including, but not limited to, groups such as naphthyl or anthryl), and includes both unsubstituted and substituted aryl groups. Substituted aryl is an aryl group that is substituted with one or more substituents, preferably one to three substituents, selected from the group consisting of alkyl, alkenyl, alkynyl, halogen, alkoxy, acyloxy, amino, mono or dialkylamino, hydroxyl, mercapto, carboxy, benzyloxy, phenyl, aryloxy, benzyl, cyano, nitro, thioalkoxy, carboxaldehyde, carboalkoxy and carboxamide, or a functionality that can be suitably blocked, if necessary for purposes of the invention, with a protecting group. Representative examples include, but are not limited to, naphthyl, phenyl, chlorophenyl, iodophenyl, methoxyphenyl, carboxyphenyl, and the like.

The term “arylene” refers to the diradical derived from aryl (including substituted aryl) as defined above and is exemplified by 1,2-phenylene, 1,3-phenylene, 1,4-phenylene, 1,2-naphthylene and the like.

The term “amino” refers to the group —NH₂.

The term “thioalkoxy” means a radical —SR where R is an alkyl as defined above e.g., methylthio, ethylthio, propylthio, butylthio, and the like. The term “mono and “dialkylamino” means a radical —NHR and —NRR′ respectively where R and R′ independently represent an alkyl group as defined herein. Representative examples include, but are not limited to dimethylamino, methylethylamino, di(1-methylethyl)amino, (cyclohexyl)(methyl)amino, (cyclohexyl)(ethyl)amino, (cyclohexyl)(propyl)amino, (cyclohexylmethyl)(methyl)-amino, (cyclohexylmethyl)(ethyl)amino, and the like.

The term “acyloxy” means a radical —OC(O)R, where R is hydrogen, alkyl, aryl, heteroaryl or substituted alkyl wherein alkyl, aryl, heteroaryl, and substituted alkyl are as defined herein. Representative examples include, but are not limited to formyl, acetyloxy, cylcohexylcarbonyloxy, cyclohexylmethylcarbonyloxy, benzoyloxy, benzylcarbonyloxy, and the like. The term “heteroalkyl,” “heteroalkenyl,” and “heteroalkynyl” refers to alkyl, alkenyl, and alkynyl groups respectively as defined above, that contain the number of carbon atoms specified (or if no number is specified, having 1 to 12 carbon atoms) which contain one or more heteroatoms, preferably one to three heteroatoms, as part of the main, branched, or cyclic chains in the group. Heteroatoms are independently selected from the group consisting of —NR—, —NRR, (where each R is hydrogen or alkyl), —S—, —O—, —SR (R is hydrogen or alkyl), —OR (R is hydrogen or alkyl), and P; preferably —NR where R is hydrogen or alkyl and/or O. Heteroalkyl, heteroalkenyl, and heteroalkynyl groups may be attached to the remainder of the molecule either at a heteroatom (if a valence is available) or at a carbon atom. Examples of heteroalkyl groups include, but are not limited to, groups such as —O—CH₃, —CH₂—O—CH₃, —CH₂—CH₂—O—CH₃, —S—CH₂—CH₂—CH₃, —CH₂—CH(CH₃)—S—CH₃, —CH₂—CH₂—NH—CH₂—CH₃, 1-ethyl-6-propylpiperidino, 2-ethylthiophenyl, piperazino, pyrrolidino, piperidino, morpholino, and the like. Examples of heteroalkenyl groups include, but are not limited to, groups such as —CH═CH—NH—CH(CH₃)—CH₃, and the like.

The term “carboxaldehyde” means —CHO.

The term “carboalkoxy” means —C(O)OR where R is alkyl as defined above and include groups such as methoxycarbonyl, ethoxycarbonyl, and the like.

The term “carboxamide” means —C(O)NHR or —C(O)NRR′ where R and R′ are independently hydrogen or alkyl as defined above. Representative examples include groups such as aminocarbonyl, methylaminocarbonyl, dimethylaminocarbonyl, and the like.

The term “heteroaryl” or “HetAr” refers to an aromatic carbocyclic group of 3 to 9 ring atoms forming a single ring and having at least one hetero atom, preferably one to three heteroatoms including, but not limited to, heteroatoms such as N, O, P, or S, within the ring. Representative examples include, but are not limited to single ring such as imidazolyl, pyrazolyl, pyrazinyl, pyridazinyl, pyrimidinly, pyrrolyl, pyridyl, thiophene, and the like, or multiple condensed rings such as indolyl, quinoline, quinazoline, benzimidazolyl, indolizinyl, benzothienyl, and the like.

The heteroalkyl, heteroalkenyl, heteroalkynyl and heteroaryl groups can be unsubstituted or substituted with one or more substituents, preferably one to three substituents, selected from the group consisting of alkyl, alkenyl, alkynyl, benzyl, halogen, alkoxy, acyloxy, amino, mono or dialkylamino, hydroxyl, mercapto, carboxy, benzyloxy, phenyl, aryloxy, cyano, nitro, thioalkoxy, carboxaldehyde, carboalkoxy and carboxamide, or a functionality that can be suitably blocked, if necessary for purposes of the invention, with a protecting group. Examples of such substituted heteroalkyl groups include, but are not limited to, piperazine, pyrrolidine, morpholine, or piperidine, substituted at a nitrogen or carbon by a phenyl or benzyl group, and attached to the remainder of the molecule by any available valence on a carbon or nitrogen, —NH—SO2-phenyl, —NH—(C═O)O-alkyl, —NH—(C═O)O-alkyl-aryl, and the like. The heteroatom(s) as well as the carbon atoms of the group can be substituted. The heteroatom(s) can also be in oxidized form.

The term “heteroarylene” refers to the diradical group derived from heteroaryl (including substituted heteroaryl), as defined above, and is exemplified by the groups 2,6-pyridylene, 2,4-pyridinylene, 1,2-quinolinylene, 1,8-quinolinylene, 1,4-benzofuranylene, 2,5-pyridnylene, 2,5-indolenyl, and the like.

The term “heteroalkylene”, “heteroalkenylene”, and “heteroalkynylene” refers to the diradical group derived from heteroalkyl, heteroalkenyl, and heteroalkynyl (including substituted heteroalkyl, heteroalkenyl, and heteroalkynyl), as defined above.

The term “alkylaryl” refers to an alkyl group having the number of carbon atoms designated, appended to one, two, or three aryl groups.

The term “alkoxy” as used herein refers to an alkyl, alkenyl, or alkynyl linked to an oxygen atom and having the number of carbon atoms specified, or if no number is specified, having 1 to 12 carbon atoms. Examples of alkoxy groups include, but are not limited to, groups such as methoxy, ethoxy, tert-butoxy, and allyloxy.

The term “aryloxy” as used herein refers to an aryl group linked to an oxygen atom at one of the ring carbons. Examples of alkoxy groups include, but are not limited to, groups such as phenoxy, 2-, 3-, or 4-methylphenoxy, and the like.

The term “halogen” as used herein refer to Cl, Br, F or I substituents, preferably fluoro or chloro.

The term “—(C₁-C₁₂) alkyl, substituted alkyl, or heteroalkyl” means an alkyl, substituted alkyl or heteroalkyl group respectively as defined above and having 1 to 12 carbon atoms. For example, when R¹ is —(C₁-C₁₂) alkyl, substituted alkyl, or heteroalkyl it means that R¹ can be —(C₁-C₁₂) alkyl or —(C₁-C₁₂)substituted alkyl, or —(C₁-C₁₂)heteroalkyl.

The term “—(C₁-C₁₂) alkenyl, substituted alkenyl, or heteroalkenyl” means an alkenyl, substituted alkenyl, or heteroalkenyl group as defined above and having 1 to 12 carbon atoms.

The term “—(C₁-C₁₂) alkynyl, substituted alkynyl, or heteroalkynyl” means an alkynyl, substituted alkynyl, or heteroalkynyl group as defined above and having 1 to 12 carbon atoms.

The term “—(C₁-C₁₂) alkylene, substituted alkylene, or heteroalkylene” means an alkylene, substituted alkylene, or heteroalkylene group as defined above and having to 12 carbon atoms.

The term “—(C₁-C₁₂) alkenylene, substituted alkenylene, or heteroalkenylene” means that the alkenylene, substituted alkenylene, or heteroalkenylene group as defined above and having 1 to 12 carbon atoms.

The term “—(C₁-C₁₂) alkynylene, substituted alkynylene, or heteroalkynylene” means an alkynylene, substituted alkynylene, or heteroalkynylene group as defined above and having 1 to 12 carbon atoms.

The term “and —(C₁-C₈ alkylene or substituted alkylene)n5-(C₃-C₁₂ arylene or heteroarylene)-(C₁-C₈ alkyl or substituted alkyl)n6 where n5 and n6 are independently 0 or 1” means that “when n5 and/or n6 are 0 then —(C₁-C₈ alkylene or substituted alkylene)n5 and/or —(C₁-C₈ alkylene or substituted alkylene)n6” are a covalent bond or when n5 and/or n6 are 1, then the alkylene or substituted alkylene group is present and can have 1 to 8 carbon atoms. The term —(C₃-C₁₂ arylene or heteroarylene)- means that the arylene has 6 to 12 carbon atoms (e.g., phenylene, naphtylene, and the like) and heteroarylene groups have 3 to 12 carbons atoms and additionally contain one to three heteroatoms including, but not limited to, heteroatoms such as N, O, P, or S, within the ring (e.g., 2,6-pyridylene, 2,4-pyridinylene, 1,2-quinolinylene, 1,8-quinolinylene, 1,4-benzofuranylene, 2,5-pyridylene, 2,5-indolenyl, and the like) in accordance with the definition of the heteroarylene above. Additionally, it will be recognized by a person skilled in the art that when “—(C1-C8 alkylene or substituted alkylene)- “and” —(C1-C8 alkyl or substituted alkyl)-” are a covalent bond then —(C3-C12 arylene or heteroarylene)- is an aryl or heteroaryl group as defined above.

“Protecting group” refers to a chemical group that exhibits the following characteristics: 1) reacts selectively with the desired functionality in good yield to give a protected substrate that is stable to the projected reactions for which protection is desired; 2) is selectively removable from the protected substrate to yield the desired functionality; and 3) is removable in good yield by reagents compatible with the other functional group(s) present or generated in such projected reactions. Examples of suitable protecting groups can be found in Greene et al. (1991) Protective Groups in Organic Synthesis, 2nd Ed. (John Wiley & Sons, Inc., New York). Preferred amino protecting groups include, but are not limited to, benzyloxycarbonyl (CBz), t-butyl-oxycarbonyl (Boc), t-butyldimethylsilyl (TBDIMS), 9-fluorenylmethyl-oxycarbonyl (Fmoc), or suitable photolabile protecting groups such as 6-nitroveratryloxy carbonyl (Nvoc), nitropiperonyl, pyrenylmethoxycarbonyl, nitrobenzyl, dimethyl dimethoxybenzil, 5-bromo-7-nitroindolinyl, and the like. Preferred hydroxyl protecting groups include Fmoc, TBDIMS, photolabile protecting groups (such as nitroveratryl oxymethyl ether (Nvom)), Mom (methoxy methyl ether), and Mem (methoxy ethoxy methyl ether). Particularly preferred protecting groups include NPEOC (4-nitrophenethyloxycarbonyl) and NPEOM (4-nitrophenethyloxy-methyloxycarbonyl).

“Inhibitor” refers to a compound that interferes with the interaction between a target and its respective substrate(s) or endogenous ligand(s). Target molecules include, but are not limited to, enzymes and receptors. Enzyme inhibitors have been extensively studied from kinetic and mechanistic standpoints; see, e.g., Fersht, A., Enzyme Structure and Mechanism, 2nd Ed., New York, W. H. Freeman, 1985. A useful measure of the effectiveness of a compound at inhibiting enzyme catalysis is the IC₅₀ of that compound. The IC₅₀ of a compound can determined by the equation

y=yo/(1+[In]/IC₅₀)

where y is the measured reaction velocity, yo is the reaction velocity in the absence of inhibitor, and [In] is the inhibitor concentration. Solving this equation at the inhibitor concentration [In] when y=yo/2 yields IC₅₀ of the inhibitor for the enzyme under study. Useful inhibitors have an IC₅₀ equal to or less than about 10 TM, preferably equal to or less than about 1 TM. More preferably, the inhibitor has an IC₅₀ equal to or less than about 100 nM, still more preferably equal to or less than about 10 nM, even more preferably equal to or less than about 1 nM. Most preferably, inhibitors have an IC₅₀ equal to or less than about 100 pM, or equal to or less than about 10 pM.

“Treating” or “treatment” of a disease includes:

(1) preventing the disease, i.e. causing the clinical symptoms of the disease not to develop in a mammal that may be exposed to or predisposed to the disease but does not yet experience or display symptoms of the disease,

(2) inhibiting the disease, i.e., arresting or reducing the development of the disease or its clinical symptoms, or

(3) relieving the disease, i.e., causing regression of the disease or its clinical symptoms.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

A “antibacterial effective amount” means the amount of a compound that, when administered to a mammal for treating a bacterial infection, is sufficient to effect such treatment for the infection. The “antibacterial effective amount” will vary depending on the compound, the disease and its severity and the age, weight, etc., of the mammal to be treated.

A “pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use. A “pharmaceutically acceptable excipient” as used in the specification and claims includes both one and more than one such excipient.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

A “pharmaceutically acceptable salt” of a compound means a salt that is pharmaceutically acceptable and that possesses the desired pharmacological activity of the parent compound. Such salts include: A “pharmaceutically acceptable salt” of a compound means a salt that is pharmaceutically acceptable and that possesses the desired pharmacological activity of the parent compound. Such salts include:

(1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-napthalenesulfonic acid, 4-toluenesulfonic acid, camphorsulfonic acid, 3-phenylpropionic acid, trimethylacetic acid, tertiary butylacetic acid, lauryl sulfuric acid, gluconic acid, glutamic acid, hydroxynapthoic acid, salicylic acid, stearic acid, muconic acid, and the like; or

(2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

“Prodrug” means any compound which releases an active parent drug according to Formula (I) in vivo when such prodrug is administered to a mammalian subject. Prodrugs of a compound may be prepared by modifying functional groups present in the compound in such a way that the modifications may be cleaved in vivo to release the parent compound. Prodrugs include compounds of Formula (I) wherein a hydroxy, amino, or sulfhydryl group in compound (I) is bonded to any group that may be cleaved in vivo to regenerate the free hydroxyl, amino, or sulfhydryl group, respectively. Examples of prodrugs include, but are not limited to esters (e.g., acetate, formate, and benzoate derivatives), carbamates (e.g., N,N-dimethylamino-carbonyl) of hydroxy functional groups in compounds of Formula (I), and the like.

“Isomers” are compounds that have the same molecular formula but differ in the nature or sequence of bonding of their atoms or the arrangement of their atoms in space. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this invention may possess one or more asymmetric centers; such compounds can therefore be produced as individual (R)- or (S)-stereoisomers or as mixtures thereof. For example, if the R6 substituent in a compound of Formula (I) is 2-hydroxyethyl, then the carbon to which the hydroxy group is attached is an asymmetric center and therefore the compound of Formula (I) can exist as an (R)- or (S)-stereoisomer. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 1992).

By “antibiotic” is meant a chemical substance that has the ability to kill or inhibits the multiplication of microbes or microorganisms. Typically, an antibiotic is produced by microbes, and is harmful to other microbes. Bacteria, fungi, viruses, and protozoa are all examples of microbes. Antibiotics that are non-toxic to the host are often used as chemotherapeutic agents in the treatment of infectious diseases of man, animals, and plants. The compounds of the invention may be co-administered with one or more antibiotics.

Antibiotics exhibit activity against gram positive bacteria (gram positive agents), gram negative bacteria (gram negative agents) or are broad spectrum (i.e., agents active against both types). Examples of antibiotics that may be used include, but are not limited to, Acyclovir, Amantadine, Amikacin, Amoxicillin, Amoxicillin; Clavulanic Acid, Amphotericin B, Ampicillin, Sulbactam, Atovaquone, Azithromycin, Aztreonam, Bacitracin, Bismuth Subsalicylate, Carbenicillin, Cefaclor, Cefadroxil, Cephaloglycin, Cephaloridine, Cephalothin, Cephapirin, Cephradine, Cephahalexin, Cefamandole, Cefazolin, Cefepime, Cefixime, Cefoperazone, Cefotaxime, Cefotetan, Cefoxitin, Cefprozil, Ceftazidime, Ceftriaxone, Cefuroxime, Cephalexin, Chloramphenicol, Chloroquine, Cidofovir, Ciprofloxacin, Clarithromycin, Clindamycin, Clofazimine, Clotrimazole, Co-trimoxazole, Cycloserine, Dapsone, Demeclocycline, Dicloxacillin, Dirithromycin, Doxycycline, Erythromycin, Ethambutol, Famciclovir, Fleroxacin, Fluconazole, Flucytosine, Foscarnet, Ganciclovir, Gentamicin, Griseofulvin, Hydroxychloroquine, Imipenem, Cilastatin, Indinavir, Interferon Alfa, Isoniazid, Itraconazole, Ketoconazole, Lamivudine, Lindane, Lomefloxacin, Loracarbef, Mebendazole, Methenamine, Methicillin, Metronidazole, Mezlocillin, Miconazole, Minocycline, Mupirocin, Nafcillin, Neomycin, Nevirapine, Nitrofurantoin, Norfloxacin, Nystatin, Ofloxacin, Oxacillin, Penicillin G, Penicillin V, Pentamidine, Piperacillin, Piperacillin; Tazobactam, Polymyxin B, Primaquine, Pyrantel, Pyrazinamide, Pyrimethamine, Quinacrine, Quinine, Ribavirin, Rifabutin, Rifampin, Rimantadine, Ritonavir, Saquinavir, Silver Sulfadiazine, Spectinomycin, Stavudine, Streptomycin, Sulfacetamide, Sulfamethoxazole, Sulfisoxazole, Terbinafine, Tetracycline, Thiabendazole, Ticarcillin, Ticarcillin; Clavulanic Acid, Tobramycin, Trimethoprim, Valacyclovir, Vancomycin, Vidarabine, Zalcitabine, Zidovudine, and Zyvox. Examples of gram positive agents include, but are not limited to, Zyvox and Cephalosporins (e.g., Cefadroxil, Cefazolin, Cephalexin, Cephaloridine, Cephalothin, Cephapirin and Cephradine). Examples of gram negative agents include, but are not limited to, Aminoglycosides (e.g., Streptomycin, Gentamycin, Sisomicin, Netilmicin, Amikacin, Neomycin, Kanamycin and Tobramycin.). Examples of broad spectrum agents include, but are not limited to, Penicillins (e.g., Penicillin G, Ampicillin, Amoxicillin, Carbenicillin, Ticarcillin, Azlocillin and Mezlocillin), third generation Cephalosporins (e.g., Cefdinir, Cefixime, Cefoperazone, Cefotaxime, Cefpodoxime, Ceftazidime, Ceftibuten, Ceftizoxime and Ceftriaxone), Quinolones (e.g., Gatifloxacin, Grepafloxacin, Sparfloxacin, Tosufloxacin, Clinafloxacin, Moxifloxacin and Trovafloxacin) and Macrolides (e.g., Erythromycin, Dirithromycin and Clarithromycin).

D. Formulations

The present invention also provides pharmaceutical compositions which comprise a bioactive beta-amino acid derivative, or a pharmaceutically acceptable salt thereof, and a pharmaceutically acceptable carrier. The compositions of the invention include those in a form adapted for oral, topical or parenteral use and can be used for the treatment of bacterial infection in animals, preferably mammals, more preferably humans.

The antibiotic compounds, also referred to herein as antimicrobial. compounds, according to the invention can be formulated for administration in any convenient way for use in human or veterinary medicine, by analogy with other antibiotics. Such methods are known in the art (see, e.g., Remington's Pharmaceutical Sciences, Easton, Pa.: Mack Publishing Co.) and are not described in detail herein.

The composition can be formulated for administration by any route known in the art, such as subdermal, inhalation, oral, topical or parenteral. The compositions can be in any form known in the art, including but not limited to tablets, capsules, powders, granules, lozenges, creams or liquid preparations, such as oral or sterile parenteral solutions or suspensions. The compounds can also be administered in liposome formulations. The compounds can also be administered as prodrugs, where the prodrug administered undergoes biotransformation in the treated mammal to a form which is biologically active.

This invention includes pharmaceutical compositions which contain negamycin or deoxynegamycin, or a pharmaceutically acceptable salt, prodrug or isomer thereof, as the active ingredient, associated with pharmaceutically acceptable carriers. In making the compositions of this invention, the active ingredient is usually mixed with an excipient, diluted by an excipient or enclosed within such a carrier which can be in the form of a capsule, sachet, paper or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, it may be necessary to mill the active compound to provide the appropriate particle size prior to combining with the other ingredients. If the active compound is substantially insoluble, it ordinarily is milled to a particle size of less than 200 mesh. If the active compound is substantially water soluble, the particle size is normally adjusted by milling to provide a substantially uniform distribution in the formulation, e.g. about 40 mesh.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

The compositions are preferably formulated in a unit dosage form, each dosage containing from about 5 to about 100 mg, more usually about 10 to about 30 mg, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Preferably, the compound of formula I above is employed at no more than about 20 weight percent of the pharmaceutical composition, more preferably no more than about 15 weight percent, with the balance being pharmaceutically inert carrier(s).

The active compound is effective over a wide dosage range and is generally administered in a pharmaceutically effective amount. It will be understood, however, that the amount of the compound actually administered will be determined by a physician, in the light of the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation is then subdivided into unit dosage forms of the type described above containing from, for example, 0.1 to about 500 mg of the active ingredient of the present invention.

The tablets or pills of the present invention may be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the novel compositions of the present invention may be incorporated for administration orally or by injection include aqueous solutions, suitably flavored syrups, aqueous or oil suspensions, and flavored emulsions with edible oils such as corn oil, cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices which deliver the formulation in an appropriate manner.

The topical formulations of the present invention can be presented as, for instance, ointments, creams or lotions, solutions, salves, emulsions, plasters, eye ointments and eye or ear drops, impregnated dressings and aerosols, and can contain appropriate conventional additives such as preservatives, solvents to assist drug penetration and emollients in ointments and creams.

The formulations can also contain compatible conventional carriers, such as cream or ointment bases and ethanol or oleyl alcohol for lotions. Such carriers can be present, for example, from about 1% up to about 99% of the formulation. For example, they can form up to about 80% of the formulation.

Tablets and capsules for oral administration can be in unit dose presentation form, and can contain conventional excipients such as binding agents, for example syrup, acacia, gelatin, sorbitol, tragacanth, or polyvinylpyrollidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets can be coated according to methods well known in standard pharmaceutical practice.

Oral liquid preparations can be in the form of, for example, aqueous or oily suspensions, solutions, emulsions, syrups or elixirs, or can be presented as a dry product for reconstitution with water or another suitable vehicle before use. Such liquid preparations can contain conventional additives, such as suspending agents, for example sorbitol, methyl cellulose, glucose syrup, gelatin, hydroxyethyl cellulose, carboxymethyl cellulose, aluminium stearate gel or hydrogenated edible fats; emulsifying agents, for example lecithin, sorbitan monooleate, or acacia; non-aqueous vehicles (which can include edible oils), for example almond oil, oily esters such as glycerine, propylene glycol, or ethyl alcohol; preservatives, for example methyl or propyl p-hydroxybenzoate or sorbic acid, and, if desired, conventional flavoring or coloring agents.

For parenteral administration, fluid unit dosage forms are prepared utilizing the compound and a sterile vehicle, water being preferred. The compound, depending on the vehicle and concentration used, can be either suspended or dissolved in the vehicle or other suitable solvent. In preparing solutions, the compound can be dissolved in water for injection and filter sterilized before filling into a suitable vial or ampoule and sealing. Advantageously, agents such as a local anesthetic preservative and buffering agents can be dissolved in the vehicle. To enhance the stability, the composition can be frozen after filling into the vial and the water removed under vacuum. The dry lyophilized powder is then sealed in the vial and an accompanying vial of water for injection can be supplied to reconstitute the liquid prior to use. Parenteral suspensions are prepared in substantially the same manner except that the compound is suspended in the vehicle instead of being dissolved and sterilization cannot be accomplished by filtration. The compound can be sterilized by exposure to ethylene oxide before suspending in the sterile vehicle. Advantageously, a surfactant or wetting agent is included in the composition to facilitate uniform distribution of the compound.

The compositions can contain, for example, from about 0.1% by weight to about 99% by weight, e.g., from about 10-60% by weight, of the active material, depending on the method of administration. Where the compositions comprise dosage units, each unit will contain, for example, from about 1-500 mg of the active ingredient. The dosage as employed for adult human treatment will range, for example, from about 1 to 3000 mg per day, for instance 1500 mg per day depending on the route and frequency of administration. Such a dosage corresponds to about 0.015 to 50 mg/kg per day. Suitably the dosage is, for example, from about 5 to 20 mg/kg per day.

The following formulation examples illustrate representative pharmaceutical compositions of the present invention.

### Formulation Example 1

Hard gelatin capsules containing the following ingredients are prepared:

| | Quantity |
|---|---|
| Ingredient | (mg/capsule) |
| Active Ingredient | 30.0 |
| Starch | 305.0 |
| Magnesium stearate | 5.0 |

The above ingredients are mixed and filled into hard gelatin capsules in 340 mg quantities.

### Formulation Example 2

A tablet formula is prepared using the ingredients below:

| | Quantity |
|---|---|
| Ingredient | (mg/tablet) |
| Active Ingredient | 25.0 |
| Cellulose, microcrystalline | 200.0 |
| Colloidal silicon dioxide | 10.0 |
| Stearic acid | 5.0 |

The components are blended and compressed to form tablets, each weighing 240 mg.

### Formulation Example 3

A dry powder inhaler formulation is prepared containing the following components:

| Ingredient | Weight % |
|---|---|
| Active Ingredient | 5 |
| Lactose | 95 |

The active ingredient is mixed with the lactose and the mixture is added to a dry powder inhaling appliance.

### Formulation Example 4

Tablets, each containing 30 mg of active ingredient, are prepared as follows:

| | Quantity |
|---|---|
| Ingredient | (mg/tablet) |
| Active Ingredient | 30.0 mg |
| Starch | 45.0 mg |
| Microcrystalline cellulose | 35.0 mg |
| Polyvinylpyrrolidone | 4.0 mg |
| (as 10% solution in sterile water) | |
| Sodium carboxymethyl starch | 4.5 mg |
| Magnesium stearate | 0.5 mg |
| Talc | 1.0 mg |
| Total | 120 mg |

The active ingredient, starch and cellulose are passed through a No. 20 mesh U.S. sieve and mixed thoroughly. The solution of polyvinylpyrrolidone is mixed with the resultant powders, which are then passed through a 16 mesh U.S. sieve. The granules so produced are dried at 50° to 60° C. and passed through a 16 mesh U.S. sieve. The sodium carboxymethyl starch, magnesium stearate, and talc, previously passed through a No. 30 mesh U.S. sieve, are then added to the granules which, after mixing, are compressed on a tablet machine to yield tablets each weighing 120 mg.

### Formulation Example 5

Capsules, each containing 40 mg of medicament are made as follows:

| | Quantity |
|---|---|
| Ingredient | (mg/capsule) |
| Active Ingredient | 40.0 mg |
| Starch | 109.0 mg |
| Magnesium stearate | 1.0 mg |
| Total | 150.0 mg |

The active ingredient, starch, and magnesium stearate are blended, passed through a No. 20 mesh U.S. sieve, and filled into hard gelatin capsules in 150 mg quantities.

### Formulation Example 6

Suppositories, each containing 25 mg of active ingredient are made as follows:

| Ingredient | Amount |
|---|---|
| Active Ingredient | 25 mg |
| Saturated fatty acid glycerides to | 2,000 mg |

The active ingredient is passed through a No. 60 mesh U.S. sieve and suspended in the saturated fatty acid glycerides previously melted using the minimum heat necessary. The mixture is then poured into a suppository mold of nominal 2.0 g capacity and allowed to cool.

### Formulation Example 7

Suspensions, each containing 50 mg of medicament per 5.0 mL dose are made as follows:

| Ingredient | Amount | |
|---|---|---|
| Active Ingredient | 50.0 | mg |
| Xanthan gum | 4.0 | mg |
| Sodium carboxymethyl cellulose (11%) | 50.0 | mg |
| Microcrystalline cellulose (89%) | | |
| Sucrose | 1.75 | g |
| Sodium benzoate | 10.0 | mg |
| Flavor and Color | q.v. | |
| Purified water to | 5.0 | mL |

The active ingredient, sucrose and xanthan gum are blended, passed through a No. 10 mesh U.S. sieve, and then mixed with a previously made solution of the microcrystalline cellulose and sodium carboxymethyl cellulose in water. The sodium benzoate, flavor, and color are diluted with some of the water and added with stirring. Sufficient water is then added to produce the required volume.

### Formulation Example 8

| | Quantity |
|---|---|
| Ingredient | (mg/capsule) |
| Active Ingredient | 15.0 mg |
| Starch | 407.0 mg |
| Magnesium stearate | 1.0 mg |
| Total | 425.0 mg |

The active ingredient, starch, and magnesium stearate are blended, passed through a No. 20 mesh U.S. sieve, and filled into hard gelatin capsules in 425.0 mg quantities.

### Formulation Example 9

A subcutaneous formulation may be prepared as follows:

| Ingredient | Quantity | |
|---|---|---|
| Active Ingredient | 5.0 | mg |
| Corn Oil | 1.0 | mL |

### Formulation Example 10

A topical formulation may be prepared as follows:

| Ingredient | Quantity | |
|---|---|---|
| Active Ingredient | 1-10 | g |
| Emulsifying Wax | 30 | g |
| Liquid Paraffin | 20 | g |
| White Soft Paraffin | to 100 | g |

The white soft paraffin is heated until molten. The liquid paraffin and emulsifying wax are incorporated and stirred until dissolved. The active ingredient is added and stirring is continued until dispersed. The mixture is then cooled until solid.

a. Another preferred formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art. See, e.g., U.S. Pat. No. 5,023,252, issued Jun. 11, 1991, herein incorporated by reference. Such patches may be constructed for continuous, pulsatile, or on demand delivery of pharmaceutical agents.

Frequently, it will be desirable or necessary to introduce the pharmaceutical composition to the brain, either directly or indirectly. Direct techniques usually involve placement of a drug delivery catheter into the host's ventricular system to bypass the blood-brain barrier. One such implantable delivery system used for the transport of biological factors to specific anatomical regions of the body is described in U.S. Pat. No. 5,011,472 which is herein incorporated by reference.

Indirect techniques, which are generally preferred, usually involve formulating the compositions to provide for drug latentiation by the conversion of hydrophilic drugs into lipid-soluble drugs. Latentiation is generally achieved through blocking of the hydroxy, carbonyl, sulfate, and primary amine groups present on the drug to render the drug more lipid soluble and amenable to transportation across the blood-brain barrier. Alternatively, the delivery of hydrophilic drugs may be enhanced by intra-arterial infusion of hypertonic solutions which can transiently open the blood-brain barrier.

Other suitable formulations for use in the present invention can be found in Remington's Pharmaceutical Sciences, Mace Publishing Company, Philadelphia, Pa., 17th ed. (1985), which is hereby incorporated in its entirety.

C. Utility

The compounds of the present invention may be used for the treatment of bacterial infections in animals, preferably in mammals, and most preferably in humans. More specifically, the beta-amino acid derivative of the invention, as well as pharmaceutically acceptable salts, prodrugs, and isomers thereof, are useful for the treatment of bacterial infections.

As noted above, the compounds described herein are suitable for use in a variety of drug delivery systems described above. Additionally, in order to enhance the in vivo serum half-life of the administered compound, the compounds may be encapsulated, introduced into the lumen of liposomes, prepared as a colloid, or other conventional techniques may be employed which provide an extended serum half-life of the compounds. A variety of methods are available for preparing liposomes, as described in, e.g., Szoka, et al., U.S. Pat. Nos. 4,235,871, 4,501,728 and 4,837,028, each of which is incorporated herein by reference.

The amount of compound administered to the patient will vary depending upon what is being administered, the purpose of the administration, such as prophylaxis or therapy, the state of the patient, the manner of administration, and the like. In therapeutic applications, compositions are administered to a patient already suffering from a bacterial infection in an amount sufficient to at least partially arrest further onset of the symptoms of the disease and its complications. An amount adequate to accomplish this is defined as “therapeutically effective dose.” Amounts effective for this use will depend on the judgment of the attending clinician depending upon factors such as the degree or severity of the bacterial infection in the patient, the age, weight and general condition of the patient, and the like. Preferably, for use as therapeutics, the compounds described herein are administered at dosages ranging from about 0.1 to about 500 mg/kg/day.

a. In prophylactic applications, compositions are administered to a patient at risk of developing a bacterial infection in an amount sufficient to inhibit the onset of symptoms of the disease. An amount adequate to accomplish this is defined as “prophylactically effective dose.” Amounts effective for this use will depend on the judgment of the attending clinician depending upon factors such as the age, weight and general condition of the patient, and the like. Preferably, for use as prophylactics, the compounds described herein are administered at dosages ranging from about 0.1 to about 500 mg/kg/day.

b. As noted above, the compounds administered to a patient are in the form of pharmaceutical compositions described above. These compositions may be sterilized by conventional sterilization techniques, or may be sterile filtered. When aqueous solutions are employed, these may be packaged for use as is, or lyophilized, the lyophilized preparation being combined with a sterile aqueous carrier prior to administration. The pH of the compound preparations typically will be between 3 and 11, more preferably from 5-9 and most preferably from 7 and 8. It will be understood that use of certain of the foregoing excipients, carriers, or stabilizers will result in the formation of pharmaceutical salts.

The following synthetic and biological examples are offered to illustrate this invention and are not to be construed in any way as limiting the scope of this invention. Unless otherwise stated, all temperatures are in degrees Celsius.

### EXAMPLES

The abbreviations in the examples below have their generally acceptable meaning.

In the examples below, all temperatures are in degrees Celsius (unless otherwise indicated) and the following procedures were used to prepare the compounds as indicated.

A. Preparation of Compounds

1. General Synthetic Procedures

Method A: To a stirred solution of aspartic acid derivative 1 (76 mmol) in tetrahydrofuran (200 mL) at −15° C., was added N-methylmorpholine (8 g, 76 mmol) followed by dropwise addition of isobutyl chloroformate (10 g, 76 mmol) over 30 min period. The reaction mixture was continued stirring for 30 minutes at −15° C. The white precipitate was filtered off and was washed quickly with tetrahydrofuran (40 mL). The combined filtrate was added dropwise, within 10 minutes, to a suspension of sodium borohydride (5.6 g, 152 mmol) in water (30 mL) at 0-5° C. After the completion of addition, the reaction mixture was left stirring at room temperature for 2 h. The reaction mixture was acidified with 0.5 N aqueous hydrochloric acid solution and the aqueous layer was extracted with ethyl acetate (2×200 mL). The combined organic layer was washed with saturated bicarbonate (200 mL) followed by brine (200 mL). The organic layer was dried (MgSO₄), filtered, and concentrated in vacuo. The residue was purified by silica gel column chromatography to give the alcohol. (See Scheme 1.)

Method B: To a stirred solution of oxalyl chloride (3.55 mL, 66.65 mmol) in anhydrous dichloromethane (50 mL) at −60° C., a solution of anhydrous dimethyl sulfoxide (6 mL) in anhydrous dichloromethane (30 mL) was added dropwise. After completion of the addition, the reaction mixture was stirred for 5 minutes and then a solution of alcohol (36.31 mmol), prepared above, in dichloromethane (50 mL) was added dropwise. The reaction mixture was continued stirring for further 15 minutes at −60° C. and then added triethylamine (25 mL, 179 mmol) dropwise. The reaction temperature was maintained at −60° C. for an additional 1 h during which period TLC analysis showed complete consumption of starting material. The reaction mixture was then diluted with dichloromethane (100 mL) and washed with brine (50 mL), followed by 0.5 N aqueous hydrochloric acid solution (50 mL). The organic layer was dried (MgSO₄) and the solvent was removed in vacuo to give the aldehyde 2. (See Scheme 1.)

Method C: To a stirred solution of triethyl 2-fluoro-2-phosphonoacetate (12 mL, 60 mmol) in anhydrous tetrahydrofuran (30 mL) under nitrogen atmosphere at −78° C. was added n-butyllithium (21.6 mL, 2.5 M solution in hexane) dropwise. The reaction mixture was stirred for 30 minutes, and a solution of aldehyde 2 (36 mmol) in tetrahydrofuran (30 mL) was added dropwise. The resultant reaction mixture was left stirring at −78° C. for 2 h. The reaction mixture was acidified with hydrochloric acid (0.5 N, 50 mL) and then extracted with ethyl acetate (3×100 mL). The combined organic layer was washed with brine and dried (MgSO₄). Removal of the solvent in vacuo gave the residue, which was purified by silica gel column chromatography to give pure olefin 3. (See Scheme 1.)

Method D: To a solution of olefin 3 (24 mmol) in ethyl acetate (150 mL) was added palladium on charcoal (10%, 2.0 g). The resultant reaction mixture was hydrogenated at 50 psi for a period of 16 h. The catalyst was filtered through a pad of celite and washed with ethyl acetate. The combined filtrate was concentrated in vacuo to give the pure ester 4. (See Scheme 1.)

Method E: To a stirred solution of ester 4 (24 mmol) in a mixture of tetrahydrofuran (60 mL) and methanol (10 mL) at 0° C., was added sodium borohydride (1.8 g, 48 mmol) and kept stirring for 2 h. To the reaction mixture was added ethyl acetate (50 mL) and this was acidified by pouring into a ice cold aqueous hydrochloric acid (100 mL, 0.5 N), which was then extracted with ethyl acetate (3×100 mL). The combined organic layer was dried (MgSO₄), filtered, and the solvent was removed in vacuo to give the residue, which purified by silica gel column to give the pure alcohol. (See Scheme 1.)

Method F: To a stirred mixture of alcohol (33 mmol), prepared above, and triethylamine (11.5 mL, 82 mmol) in anhydrous dichloromethane (75 mL) at 0° C., was added methanesulfonyl chloride (5.10 mL, 66 mmol) dropwise and continued stirring at 0° C. for additional 2 h. The reaction mixture was poured into water (200 mL) and extracted with ethyl acetate (2×100 mL). The combined organic layer was washed with aqueous hydrochloric acid (100 mL, 0.5 N), brine (100 mL), and then dried (Na₂SO₄). The mesylate, obtained on removal of solvent, was used in the next step without further purification. (See Scheme 1.)

Method G: To a stirred solution of mesylate (14 g, 35 mmol), prepared above, in anhydrous N,N-dimethylformamide (35 mL) was added sodium azide (9.12 g, 0.14 moles) and the resultant reaction mixture was kept stirring at 85° C. for 16 h. The solvent was removed under reduced pressure, the residue was suspended in water (100 mL) and then extracted with ethyl acetate (2×100 mL). The combined organic layer was washed with brine (100 mL), dried (MgSO₄), and the solvent was removed in vacuo to give the azide as a diastereomeric mixture. (See Scheme 1.)

Method H: To the azido ester (12 mmol), prepared in the above step, was added methanolic sodium hydroxide solution (2 M, 30 mL) and the resultant reaction mixture was stirred at room temperature overnight. Methanol was removed in vacuo and the residue was taken in water (100 mL). This was extracted with ethyl acetate (50 mL) and discarded the organic layer. The aqueous layer was acidified with 1 N aqueous hydrochloric acid solution and then extracted with ethyl acetate (3×100 mL). The combined organic layer was dried (NaSO₄) and then the solvent was removed in vacuo to give the azido acid 5. (See Scheme 1.)

Method I: To a solution of protected amino acid 6 (8.6 g, 40 mmol) in anhydrous tetrahydrofuran (120 mL) at −15° C. was added N-methylmorpholine followed by dropwise addition of isobutyl chloroformate (5.7 mL, 44 mmol). The reaction mixture was stirred at −15° C. for 30 min and then it was brought to 0° C. To this was added ethereal solution of diazomethane (prepared from diazald 42.8 g, 200 mmol). The resultant reaction mixture was stirred at room temperature for 4 h. The excess diazomethane was decomposed by dropwise addition of acetic acid and diluted the reaction mixture with ether (400 mL). This was washed with brine (2×100 mL), saturated aqueous sodium bicarbonate (2×100 mL), saturated aqueous ammonium chloride (2×100 mL), and finally with brine (100 mL). The organic layer was dried (MgSO₄) and the solvent was removed in vacuo. The residue was used immediately in the next step without purification. (See Scheme 2.)

To a stirred solution of diazoketone, prepared above, in anhydrous methanol or t-butanol (200 mL round bottom flask wrapped with aluminum foil) was added a solution of silver benzoate (600 mg) in triethylamine (6 mL, exothermic reaction) and continued stirring at room temperature overnight. The brown solid was filtered through a pad of celite and the filtrate was concentrated in vacuo. The resultant residue was dissolved in ether (400 mL), washed with water (3×100 mL), dried the organic layer (MgSO₄), and then concentrated in vacuo. The residue was purified by column chromatography to give homolagated acid as a methyl t-butyl ester derivative. (See Scheme 2.)

Method J: To a solution of methyl ester or δ-lactone (15.20 mmol) in methanol (30 ml) at 0° C. was added 1 N lithium hydroxide solution (35 mL). The reaction mixture was stirred at room temperature for 14 h, the solvent was removed in vacuo, and then the residue redissolved in water (50 mL). This was extracted with ether (2×50 mL) and discarded the organic layer. Acidified the aqueous layer with 1 N hydrochloric acid at 0° C. and extracted with 1:1 mixture of ether/ethyl acetate (3×75 mL), after saturating the aqueous layer with sodium chloride. The combined organic layer was dried (MgSO₄) and then removal of the solvent in vacuo gave the alkenoic or hydroxy acid. (See Scheme 2.)

Method K: To a stirred solution of sodium bicarbonate (15 g, 176.5 mmol) in water (125 mL) was added alkenoic acid 7 (26.16 mmol) and dichloromethane (200 mL). This was cooled to 0° C. and added a solution of potassium iodide (15 g, 90 mmol) solution in water (20 mL) followed by solid iodine (12.5 g, 49.40 mmol). After completion of the addition, cold bath was removed and the reaction mixture was continued stirring at room temperature for 2.5 h. The reaction mixture was diluted with dichloromethane (300 mL) and the organic layer was separated. The aqueous layer was further extracted with dichloromethane (100 mL). The combined organic layer was washed with dilute solution of potassium hydrogensulfate, dried (MgSO₄), the solvent was removed in vacuo, and the residue was used in the next step immediately without further purification. (See Scheme 2)

Method L: To a stirred solution of iodo lactone, product from the above step, in anhydrous N,N-dimethylformamide (50 mL) was added sodium azide (5 g) and the resultant reaction mixture was kept stirring at 60° C. for 10 h. N,N-dimethylformamide was removed in vacuo and the residue was taken in 1:1 mixture of ethyl acetate and ether (300 mL). This was washed with water (3×100 mL), the organic layer was dried (MgSO₄) and concentrated in vacuo. The residue was purified by column chromatography to give the azido lactone 8. (See Scheme 2.)

Method M: To a solution of hydroxy acid, prepared above, in anhydrous dichloromethane (100 mL) was added imidazole (4 g, 5.95 mmol). This was cooled to 0° C. and then added t-butyldimethylsilyl chloride (4.5 g, 29.85 mmol). The reaction mixture was stirred at 0° C. for 20 min and then at room temperature for 18 h. The reaction mixture was diluted with dichloromethane (150 mL) and washed with water (2×100 mL). The organic layer was dried (MgSO₄) and the solvent was removed in vacuo to give silylated derivative which was used in the next step without further purification. (See Scheme 2.)

Method N: To a solution of silyl ester, prepared above, in methanol (50 mL) was added a solution of potassium carbonate (1.93 g, 13.86 mmol) in water (20 mL). The reaction mixture was stirred at room temperature for 2 h and then methanol was removed in vacuo. The residue was diluted with water (100 mL), extracted with ether (2×30 mL), and discarded the ether layer. The aqueous layer was neutralized with 0.5 N hydrochloric acid at 0° C. This was extracted with 1:1 mixture of ethyl acetate and ether (3×100 mL). The combined organic layer was dried (MgSO₄) and then removal of solvent in vacuo gave the 6-azidohexanoic acid. (See Scheme 2.)

Method O: To a solution of 6-azidohexanoic acid (, 4.47 mmol), from above step, in anhydrous dichloromethane (20 mL) and anhydrous pyridine (5 mL, 61.82 mmol) was added pentafluorophenyl trifluoroacetate (0.92 mL, 5.34 mmol). The resultant reaction mixture was stirred at room temperature for 24 h and then diluted with dichloromethane (100 mL). This was washed with ice cold 0.3 N hydrochloric acid (3×75 mL), ice cold 0.1 N sodium hydroxide solution (3×75 mL), the organic layer was dried (MgSO₄). The solvent was concentrated in vacuo to give pentfluorophenyl 9, as a diastereomeric mixture. (See Scheme 2.)

Method P: To a stirred mixture of acid 5 or 14 (3.94 mmol), amine 13 (4.73 mmol) and HATU (1.79 g, 4.73 mmol) in anhydrous N,N-dimethylformamide (10 mL) at 0° C. was added diisopropylethylamine (2.47 mL, 14.2 mmol) and continued stirring at 0° C. for 1 h. After stirring the reaction mixture overnight at ambient temperature, N,N-dimethylformamide was removed in vacuo. The residue was taken in ether (300 mL), washed with water (100 mL), the organic layer was dried (MgSO₄), and then removal of solvent in vacuo gave the residue which was purified by column chromatography to give the desired hydrazide 10. (See Scheme 3.)

Method Q: To a solution of pentafluorophenyl ester 9 (0.88 mmol) in anhydrous N,N-dimethylformamide (1 mL) was added amine 13 (1.42 mmol) and the mixture was stirred at room temperature for 3 h. Removed the solvent in vacuo, the residue was taken in ether (75 mL) and washed with 5% sodium hydroxide solution (2×20 mL). The organic layer was dried (MgSO₄), concentrated in vacuo, and the resultant residue was purified by column chromatography to give the desired hydrazide 10. (See Scheme 3.)

Method R: To a stirred solution of azide 10 (0.23 mmol) in ethyl acetate or methanol (1.8 mL) was added Pd-C (15 mg, 5%) and the resultant reaction mixture was subjected to catalytic hydrogenation using a balloon full of hydrogen at room temperature for 3 h. The catalyst was filtered through a pad of celite and was washed with ethyl acetate (10 mL). The combined filtrate was concentrated in vacuo and the residue was purified by passing through the ion exchange column (Mega bound elut SCX column, sulfonic acid on silica gel). The column was initially eluted with methanol to remove non-basic impurities and then eluted with ethanolic ammonia (1 M solution) to get the desired amine. Solvent was removed in vacuo, the residue was taken in water (3 mL), and was lyophilized to give the amine 11. (See Scheme 3.)

Method S: To a stirred solution of azide 10 (0.1 g, 0.19 mmol) in tetrahydrofuran (1.5 mL) was added triphenylphosphine (0.12 g, 0.23 mmol) and continued stirring at ambient temperature for 12 h. To this was added water (1 mL) and tetrahydrofuran (1.5 mL), continued stirring at ambient temperature for 4 h, and then warmed to 50° C. for 4 h. The solvent was removed in vacuo and then extracted with ether (4×5 mL). The combined organic layer was dried (MgSO₄), concentrated in vacuo, and the residue was purified by passing through the ion exchange column (Mega bound elut SCX column, sulfonic acid on silica gel). The column was initially eluted with methanol to remove non basic impurities and then eluted with ethanolic ammonia (1 M solution) to get the desired amine. Solvent was removed in vacuo, the residue was taken in water (3 mL), and was lyophilized to give the desired amine 11. (See Scheme 3.)

Method T: The protected amine 11 (0.198 mmol) was taken in 4 N hydrochloric acid in dioxane (1.5 mL) and then added 3 drops of water (slightly exothermic). The resultant solution was stirred at room temperature for 5 h and then the solvent was removed in vacuo. The residue was dissolved in water (5 mL) and extracted with ether (2×5 mL). The aqueous layer was lyophilized to give the fully deprotected product 12 as a hydrochloride salt. (See Scheme 3.)

Method U: The protected amine 11 (0.065 mmol) was dissolved in a mixture of trifluoroacetic acid and dichloromethane (30%, 0.5 mL) and stirred at room temperature for 4 h. The solvent was removed in vacuo and the residue was dried in vacuo. This was dissolved in a mixture of tetrahydrofuran (0.3 mL) and pyridine (0.1 mL) and cooled to 0° C. To this was added hydrogen fluoride in pyridine (0.1 mL) and continued stirring at 0° C. for 30 min and then at room temperature for 1.5 h. The reaction mixture was diluted with water (10 mL) and lyophilized to get the product, which was purified by ion exchange column (CG 50, initially eluted with water followed by 1% ammonium hydroxide) to give the desired compound as aammonium salt. (See Scheme 3.)

Method V: Collidine (57 mg, 0.47 mmol) was added to a solution of amine 11a (0.39 mmol) and 2-nitrophenylsulfonyl chloride (94 mg, 0.43 mmol) in dichloromethane (0.2 mL) at 4° C. and the mixture was warmed to 23° C. After 8 h, the mixture was diluted with ethyl acetate (2 mL), washed with 5% aqueous potassium hydrogensulfate (2×1 mL), brine (2×1 mL), and dried the organic layer (Na₂SO₄). The solvent was removed in vacuo and the residue was purified by column chromatography to give the sulfonamide 17. (See Scheme 5.)

Method W: A mixture of cesium carbonate (24 mg, 0.075 mmol) and sulfonamide 17 (35 mg, 0.05 mmol) in N,N-dimethylformamide (0.5 mL) was stirred at ambient temperature for 30 min. The alkyl halide (0.075 mmol) was added and the reaction mixture was stirred for 2 h at ambient temperature. The mixture was diluted with ethyl acetate (5 mL), washed with brine (3×2mL), dried the organic layer (Na₂SO₄) and evaporated in vacuo to give the alkylated sulfonamide 18. (See Scheme 5.)

Method X: To a stirred solution containing sulfonamide 17 (50 mg, 0.071 mmol), triphenylphosphine (24 mg, 0.090 mmol) and 4-methyl-1-pentanol (9 mg, 0.090 mmol) in dichloromethane (0.07 mL) at 4° C. was added DIAD (17 mg, 0.085 mmol) over 10 min. The reaction temperature was raised to 23° C. for 3 h, then the mixture was concentrated in vacuo. The residue was purified by flash silica gel chromatography to give the alkylated sulfonamide 18. (See Scheme 5.)

Method Y: A solution of sulfonamide 18 (0.047 mmol) and thiophenol (7 mg, 0.061 mmol) in N,N-dimethylforamide (0.4 mL) was treated with DBU (7.9 mg, 0.052 mmol) at room temperature for 12 h. The solution was diluted with diethyl ether (4 mL), washed with dilute aqueous sodium carbonate solution (2×1 mL), brine (2×1 mL), the organic layer was dried (Na₂SO₄), and concentrated in vacuo. The residue was purified by silica gel or ion exchange column chromatography. (See Scheme 5.)

Method Z: A mixture of aldehyde 20 (92 mmol), malonic acid (19.1 g, 184 mmol) and ammonium acetate (28.3 g, 367 mmol) in ethanol (50 mL) was heated to reflux for 24 h with constant stirring. Ethanol was removed in vacuo and the residue was suspended in water (200 mL). The precipitate was collected by filtration and the aqueous portion was mixed with saturated sodium bicarbonate solution (150 mL). This was extracted with ethyl acetate (200 mL) and discarded the organic layer. The aqueous layer was acidified with hydrochloric acid (10%, 200 mL), extracted with ethyl acetate (2×100 mL) and discarded the organic layer. The aqueous layer was concentrated in vacuo to obtain a yellow solid, which was suspended in methanol (100 mL) and filtered to remove the insoluble solid. The filtrate was concentrated to obtain b-amino acid 22. (See Scheme 6.)

Method AA: To a stirred solution of amino acid 23 (5 mmol) in a mixture of dioxane (25 mL) and water (25 mL) was added sodium carbonate or sodium hydroxide (2.5 g, 29 mmol) followed by di-t-butyl dicarbonate (4.4 g, 20 mmol) and continued stirring at room temperature for 2 h. The reaction mixture was diluted with water (20 mL), extracted with ethyl ether (50 mL), and discarded the organic layer. The aqueous layer was acidified with 1 N HCl (30 mL) at 0° C. and extracted with ethyl acetate (2×50 mL). The combined organic layer was washed with brine (30 mL), dried (MgSO₄), and concentrated in vacuo to give the protected b-amino acid 24. (See Scheme 6.)

Method BB: To a solution of amine hydrochloride 26 (36 mg, 0.13 mmol) in a mixture of water (10 ml) and AcOH (10 mL) was added PtO₂ (20 mg, 0.09 mmol) and then the reaction mixture was subjected to hydrogenation at 50 psi for 24 h. The catalyst was filtered off and the filtrate was concentrated in vacuo. The residue was dissolved in water (10 mL) and lyophilized to provide the amine as an acetate salt. The acetate salt was purified on ion exchange column (Amberlite CG-50 resin) by eluting with 2% ammonium hydroxide to give pure ammonium salt. (See Scheme 6.)

2. Synthetic Procedures for Preferred Compounds

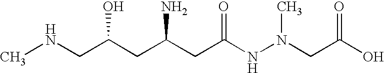

Step 1: Methyl (3R)-t-butoxycarbonylaminohex-5-enoate was prepared from N-t-butyloxycarbonyl-D-allylglycine (6, R²=R³=R⁴=R⁶=R⁷=H) in 76% yield according to the Method I, after purification by column chromatography using 9:1 hexanes/ethyl acetate mixture as an eluent. R_{f}=0.13 (9:1 hexanes/ethyl acetate, silica). ¹H NMR (300 MHz, CDCl₃) δ1.42 (s, 9H), 2.30 (bt, J=6.6 Hz, 2H), 2.52 (d, J=5.4 Hz, 2H), 3.68 (s, 3H), 4.08 (bs, 1H) 4.94 (bs, 1H), 5.01 (m, 2H), 5.80 (m, 1H). MS (m/z): 244 (M+H).

Step 2: (3R)-t-butoxycarbonylaminohex-5-enoic acid (7, R²=R³=R⁴=R⁶=R⁷=H) was prepared from methyl (3R)-t-butoxycarbonylaminohex-5-enoate according to the Method J in quantitative yield and used in the next step without further purification. R_{f}=0.17 (5% methanol in dichloromethane). ¹H NMR (300 MHz, CDCl₃) δ1.44 (s, 9H), 2.35 (bt, J=7.2 Hz, 2H), 2.59 (bs, 2H), 3.99 (bs, 1H), 4.95 (bs, 1H), 5.13 (m, 2H), 5.78 (m, 1H). MS (m/z): 252 (M+Na).

Step 3: [(2R/S)-Iodomethyl-6-oxo-tetrahydropyran-4-yl]carbamic acid t-butyl ester was prepared from (3R)-t-butyloxycarbonylaminohex-5-enoic acid (7, R²=R³=R⁴=R⁶=R⁷=H) according to Method K and the residue was used in the next step immediately. R_{f}=0.25 (1:1 hexanes/ethyl acetate).

Step 4: [(2R/S)-Azidomethyl-6-oxo-tetrahydropyran-4-yl]carbamic acid t-butyl ester (8, R²=R³=R⁴=R⁶=R⁷=H) was synthesized from [(2R/S)-iodomethyl-6-oxo-tetrahydropyran-4-yl]carbamic acid t-butyl ester following the Method L in 62% yield, after purification by silica gel column chromatography using hexanes/ethyl acetate (3:2) as an eluent. Rₜ=0.19 (1:1 hexanes/ethyl acetate). ¹H NMR (300 MHz, CDCl₃) δ1.44 (s, 9H), 1.93-2.41 (m, 2H), 2.57-3.01 (m, 2H), 3.41-3.61 (m, 2H), 4.10-4.15 (m, 1H), 4.38-4.56 (m, 1H) 4.64-4.72 (m, 1H). ¹³C NMR (75 MHz, CDCl₃) d 23.76, 25.94, 27.74, 31.02, 32.04, 38.12, 38.91, 49.41, 49.61, 70.49, 71.93, 75.43, 150.40, 150.65, 164.79. MS(m/z): 270 (M), 293 (M+Na).

Step 5: A solution of [(2R/S)-azidomethyl-6-oxo-tetrahydropyran-4-yl]carbamic acid t-butyl ester (8, R²=R³=R⁴=R⁶=R⁷=H, 3.7 g, 14 mmol in methanol (50 mL) was subjected to base hydrolysis according to the Method J to obtain 6-azido-(3R)-t-butoxycarbonylamino-(5R/S)-hydroxyhexanoic acid and used in the next step without further purification. MS (m/z): 289 (M+H).

Step 6: t-Butyldimethylsilyl 6-azido-(3R)-t-butoxycarbonylamino-(5R/S)-t-butyldimethylsilanyloxyhexanate was prepared from 6-azido-(3R)-t-butoxycarbonylamino-(5R/S)-hydroxyhexanoic acid according to the Method M and used the crude product in the next step without further purification.

Step 7: 6-Azido-(3R)-t-butoxycarbonylamino-(5R/S)-t-butyldimethylsilanyloxyhexanoic acid was synthesized from t-butyldimethylsilyl 6-azido-(3R)-t-butoxycarbonylamino-(5R/S)-t-butyldimethylsilanyloxyhexanate following the Method N in 80% yield and used in the next step without further purification. R_{f}=0.2 (5% methanol in dichloromethane). ¹H NMR (300 MHz, CDCl₃) δ0.0 (s, 3H), 0.02 (s, 3H), 0.82 (s, 9H), 1.35 (s, 9H), 1.61 (bs, 2H) 2.54 (bs, 2H), 3.12-3.40 (m, 2H), 3.81 (bm, 2H), 4.98 (bs, 1H). MS (m/z): 403 (M+H).

Step 8: Pentafluorophenyl 6-azido-(3R)-t-butoxycarbonylamino-(5R/S)-t-butyldimethylsilanyloxyhexanate (9, R²=R³=R⁴=R⁶=R⁷=H) was synthesized from 6-azido-(3R)-t-butoxycarbonylamino-(5R/S)-t-butyldimethylsilanyloxyhexanoic acid according Method O in 79% yield. The diastereomeric mixture was separated by column chromatography using 10:1 hexane/ethyl acetate. Isomer A (HR_{f}) Rₜ=0.16 (9:1 hexanes/ethyl acetate, Plate was developed two times). ¹H NMR (300 MHz, CDCl₃) δ0.01 (s, 3H), 0.02, (s, 3H), 0.89 (s, 9H), 1.44 (s, 9H), 1.73-1.94 (m, 2H), 2.8-2.93 (m, 2H), 3.08-3.34 (m, 2H), 3.78-3.96 (m, 2H), 4.83-4.98 (m, 1H). MS m/z): 569 (M+H). Isomer B (LR_{f}) Rₜ=0.14 (9:1 hexanes/ethyl acetate, plate was developed two times). ¹H NMR (300 MHz, CDCl₃) δ0.01 (s, 3H), 0.02, (s, 3H), 0.81 (s, 9H), 1.33 (s, 9H), 1.73 (bm, 2H), 2.78-2.98 (m, 2H), 3.07-3.28 (m, 2H), 3.84-3.87 (m, 1H), 3.83 (m, 1H), 4.02 (bm, 1H), 4.85 (bd, 1H). MS (m/z): 569 (M+H).

Step 9: To a stirred solution of N-methylhydrazine (3.45 mL, 75 mmol) and triethylamine (14.8 mL, 110 mmol) in dichloromethane (250 mL) at 0° C. was added a solution of t-butyl-2-bromoacetate (11.07 mL, 75 mmol) in dichloromethane (125 mL) over a period of 30 min. This was continued stirring at 0° C. for 2 h and then at ambient temperature for 14 h. The reaction mixture was concentrated in vacuo and the precipitated salt was filtered off and was washed with ether. The combined filtrate was concentrated in vacuo to get the crude product. This was purified by flash column chromatography using ethyl acetate and methanol mixture (95:5) to give the pure t-butyl (N-methylhydrazino)acetate (9.5 g, 79%). R_{f}=0.3 (5% methanol in ethyl acetate). ¹H NMR (300 MHz, CDCl₃) δ1.48 (s, 9H), 2,62 (s, 3H), 3.32 (s, 2H). MS (m/z): 161 (M+H).

Step 10: The t-butyl {N′-[6-azido-(3R)-t-butoxycarbonylamino-(5R)-t-butyldimethylsilanyloxyhexanoyl]-N-methylhydrazino}acetate (10, R²=R³=R⁴=R⁶=R⁷=R¹¹=R¹²=H, R⁵=OTBDMS, R¹³=CH₃, X=N, R¹⁴=CH₂CO₂-t-Bu) was prepared using pentafluorophenyl 6-azido-(3R)-t-butoxycarbonylamino-(5R)-t-butyldimethylsilanyloxyhexanate (9, R²=R³=R⁴=R⁶=R⁷=H) and t-butyl (N-methylhydrazino)acetate according to the Method Q in 94% yield, after purification by column chromatography using 5% methanol in dichloromethane as an eluent. R_{f}=0.37 (5% methanol in dichloromethane, silica gel). ¹H NMR (300 MHz, CDCl₃) δ0.00 (s, 6H), 0.80 (s, 9H), 1.36 (s, 9H), 1.37 (s, 9H), 1.59-1.73 (m, 2H), 2.17-2.24 (m, 2H), 2.62 (s, 3H), 3.02-3.40 (m, 2H), 3.45 (s, 2H), 3.83 (m, 2H), 5.13-5.19 (m, 1H) 7.20 (bs, 1H). MS (m/z): 545 (M+H).

Step 11: The t-butyl {N′-[6-amino-(3R)-t-butoxycarbonylamino-(5R)-t-butyldimethylsilanyloxyhexanoyl]-N-methylhydrazino}acetate (11, R²=R³=R⁴=R⁶=R⁷=R¹¹=R¹²=H, R⁵=OTBDMS, R¹³=CH₃, X=N, R¹⁴=CH₂CO₂-t-Bu) was prepared from t-butyl {N′-[6-azido-(3R)-t-butoxycarbonylamino-(5R)-t-butyldimethylsilanyloxyhexanoyl]-N-methylhydrazino}acetate (10, R²=R³=R⁴=R⁶=R⁷=R¹¹=R¹²=H, R⁵=OTBDMS, R¹³=CH₃, X=N, R¹⁴=CH₂CO₂-t-Bu) according to the Method R in 50% yield using methanol as a solvent and used in next step without further purification. ¹H NMR (300 MHz, CDCl₃) δ0.02 (s, 3H), 0.04 (s, 3H), 0.81 (s, 9H), 1.39 (s, 9H), 1.40 (s, 9H), 1.55-1.80 (m, 2H), 2.40-2.60 (m, 4H), 2.63 (s, 1.2H), 2.64 (s, 1.8H), 3.20-3.48 (m, 2H), 3.85-4.00 (m, 2H), 5.40 (s, 1H), 7.34 & 7.89 (two s, 1H). MS (m/z): 519 (M+H).

Step 12: The t-butyl {N′-[(3R)-t-butoxycarbonylamino-(5R)-t-butyldimethylsilanyloxy)-6(2-nitrobenzenesulfonylamino)hexanoyl]-N-methylhydrazino}acetate (17, R⁵=OTBDMS) was prepared from t-butyl {N′-[6-amino-(3R)-t-butoxycarbonylamino-(5R)-t-butyldimethylsilanyloxyhexanoyl]-N-methylhydrazino}acetate (11a, R⁵=OTBDMS) and 2-nitrobenzenesulfonyl chloride according to the Method V in 50% yield, after purification by column chromatography using a mixture of ethyl acetate and hexanes (2:1) as an eluent. ¹H NMR (300 MHz, CDCl₃) δ0.02 (s, 3H), 0.04 (s, 3H), 0.81 (s, 9H), 1.40 (s, 9H), 1.45 (s, 9H), 1.80 (m, 2H), 2.40-2.60 (m, 2H), 2.71 & 2.73 (two s, 3H), 3.10 (m, 2H), 3.28-3.60 (m, 2H), 3.82-4.00 (m, 2H), 5.40 (s, 1H), 7.34 (s, 0.4H), 7.72-7.89 (m, 3.6H), 8.06-8.20 (m, 1H). ¹³C NMR (75 MHz, CDCl₃) δ−4.5, −4.2, 18.3, 26.2, 28.6, 28.8, 39.9, 44.4, 45.4, 49.5, 58.7, 59.7, 68.9, 82.8 (2C), 122.0, 125.8, 125.8, 133.1, 133.9, 134.0. MS (m/z): 704 (M+H).

Step 13: The t-butyl (N′-{(3R)-t-butoxycarbonylamino-(5R)-t-butyldimethylsilanyloxy)-6-[methyl-(2-nitrobenzenesulfonyl)amino]hexanoyl}-N-methylhydrazino)acetate (18, R¹⁸=Me, R⁵=OTBDMS) was prepared from t-butyl {N′-[(3R)-t-butoxycarbonylamino-(5R)-t-butyldimethylsilanyloxy)-6(2-nitrobenzenesulfonylamino)hexanoyl]-N-methylhydrazino}acetate (17, R⁵=OTBDMS) and methyl iodide according to the Method W in 94% yield as a pale yellow oil, after purification by column chromatography using 100% ethyl acetate as an eluent. R_{f}=0.4 (100% ethyl acetate). ¹H NMR (300 MHz, CDCl₃) δ0.02 (s, 3H), 0.04 (s, 3H), 1.81 (s, 9H), 1.40 (s, 9H), 1.45 (s, 9H), 1.80 (m, 2H) 2.40-2.60 (m, 2H), 2.63 & 2.65 (two s, 3H), 2.71 & 2.73 (two s, 3H), 3.10 (m, 2H), 3.28-3.60 (m, 2H), 3.82-4.00 (m, 2H), 5.40 (s, 1H), 7.34 (s, 0.4H), 7.50-7.94 (m, 4.6H). MS (m/z): 718 (M+H).

Step 14: The sulfonamide protecting group in t-butyl (N′-{(3R)-t-butoxycarbonylamino-(5R)-t-butyldimethylsilanyloxy)-6-[methyl-(2-nitrobenzenesulfonyl)amino]hexanoyl}-N-methylhydrazino)acetate (18, R¹⁸=Me, R⁵=OTBDMS was removed using the Method Y to get the t-butyl {N′-[(3R)-t-butoxycarbonylamino-(5R)-t-butyldimethylsilanyloxy-6-methylaminohexanoyl]-N-methylhydrazino}acetate in 77% yield, after passing through a short plug of silica gel using 5% methanol/dichloromethane/0.1% triethylamine as an eluent. The resultant oil was used in the next step without further characterization.

Step 15: The {N′-[(3R)-amino-(5R)-hydroxy-6-methylaminohexanoyl)]-N-methylhydrazino}acetic acid (19, R¹⁸=Me, R⁵=OH) was prepared according to the Method T in 70% yield. ¹H NMR (300 MHz, D₂O) δ1.89-2.01 (m, 2H), 2.73 (m, 2H), 2.77 (s, 3H), 2.84 (s, 3H), 3.18 (m, 2H), 3.74 (s, 2H), 3.95 (m 1H), 4.24 (m, 1H). ¹³C NMR (75 MHz, D₂O) δ63.6, 58.6, 53.9, 46.1, 44.6, 35.8, 35.7, 33.2. MS (m/z): 261.5 (M−H).

### Example 2

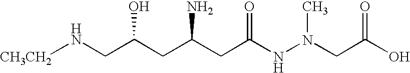

Step 1: The t-butyl (N′-{(3R)-t-butoxycarbonylamino-(5R)-t-butyldimethylsilanyloxy-6-[ethyl-(2-nitrobenzenesulfonyl)amino]hexanoyl}-N-methylhydrazino)acetate (18, R¹⁸=Et, R⁵=OTBDMS) was prepared from t-butyl {N′-[(3R)-t-butoxycarbonylamino-(5R)-t-butyldimethylsilanyloxy-6-(2-nitrobenzenesulfonylamino)hexanoyl}-N-methylhydrazino}acetate (17, R⁵=OTBDMS) and ethyl iodide following the Method W in 98% yield. R_{f}=0.27 (5% MeOH/CH₂Cl₂). ¹H NMR (300 MHz, CDCl₃) δ8.03 (m, 1H), 7.87 (s, 0.5H), 7.68 (m, 2H), 7.60 (m, 1H), 7.34 (s, 0.5H), 5.29 (s, 1H), 4.00-3.82 (m, 2H), 3.60-3.28 (m, 6H), 2.76 (s, 1.5H), 2.70 (s, 1.5H), 2.36 (m, 1H), 1.85 (m, 1H) 1.63 (m, 2H), 1.45 (s, 9H), 1.40 (s, 9H), 1.05 (t, 3H), 0.81 (s, 9H), 0.05 (s, 3H), 0.01 (s, 3H). MS (m/z): 754.3 [M+Na]⁺.

Step 2: The {N′-[(3R)-amino-6-ethylamino-(5R)-hydroxyhexanoyl]-N-methylhydrazino}acetic acid (19, R¹⁸=Et, R⁵=OH) was prepared from t-butyl (N-{(3R)-t-butoxycarbonylamino-(5R)-t-butyldimethylsilanyloxy-6-[ethyl-(2-nitrobenzenesulfonyl)amino]hexanoyl}-N-methylhydrazino)acetate (18, R¹⁸=Et, R⁵=OTBDMS) in two steps following Method Y and Method T in 75% yield for two steps. ¹H NMR (300 MHz, D₂O) δ4.20 (m, 1H), 3.93 (m, 1H), 3.74 (m, 2H),3.20 (m, 2H), 2.77 (s, 3H), 2.73 (m, 4H), 2.01 (m, 1H), 1.90 (m, 1H), 1.38 (t, 3H). ¹³C NMR (75 MHz, D₂O) δ63.9, 58.7, 52.0, 46.1, 44.6, 43.5, 36.1, 35.8, 10.7. MS (m/z): 275.5 [M−H]⁻.

### Example 3

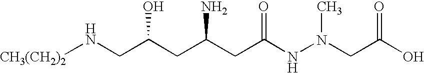

Step 1: The t-butyl (N′,-{(3R)-t-butoxycarbonylamino-(5R)-t-butyldimethylsilanyloxy-6-[(2-nitrobenzenesulfonyl)-n-propylamino]hexanoyl}-N-methylhydrazino)acetate (18, R¹⁸=n-Pr, R⁵=OTBDMS) was prepared from t-butyl {N′-[(3R)-t-butoxycarbonylamino-(5R)-t-butyldimethylsilanyloxy-6-(2-nitrobenzenesulfonylamino)hexanoyl]-N-methylhydrazino}acetate (17, R⁵=OTBDMS) and propyl iodide following the Method W in 99% yield. R_{f}=0.36 (5% MeOH/CH₂Cl₂). ¹H NMR (300 MHz, CDCl₃) δ8.03 (m, 1H), 7.87 (s, 0.5H), 7.68 (m, 2H), 7.60 (m, 1H), 7.34 (s, 0.5H), 5.29 (m, 1H), 4.00-3.82 (m 2H), 3.60-3.28 (m, 6H), 2.76 (s, 1.5H), 2.70 (s, 1.5H), 2.36 (m, 1H), 1.85 (m 1H), 1.63 (m, 4H), 1.45 (s, 9H), 1.40 (s, 9H), 0.86 (s, 9H), 0.80 (t, 3H), 0.05 (s, 3H), 0.01 (s, 3H). MS (m/z): 746.5 [M+H]⁺.

Step 2: The {N′-[(3R)-amino-(5R)-hydroxy-6-propylaminohexanoyl]-N-methylhydrazino}acetic acid (19, R¹⁸=n-Pr, R⁵=OH) was prepared from t-butyl (N′,-{(3R)-t-butoxycarbonylamino-(5R)-t-butyldimethylsilanyloxy-6-[(2-nitrobenzenesulfonyl)-n-propylamino]hexanoyl}-N-methylhydrazino)acetate (18, R¹⁸=n-Pr, R⁵=OTBDMS) in two steps following Method Y and Method T in 70% yield for two steps. ¹H NMR (300 MHz, D₂O) δ4.20 (m, 1H), 3.93 (m, 1H), 3.72 (m, 2H), 3.20-3.00 (m, 4H), 2.71 (m, 2H), 2.70 (s, 3H), 1.97 (m, 2H), 1.65 (m, 2H), 0.96 (t, 3H). ¹³C NMR (75 MHz, D₂O) δ63.9 (2C), 52.3, 49.7, 46.5, 45.1, 36.2, 36.1, 19.3, 10.5. MS (m/z): 289.6 [M−H]⁻.

### Example 4

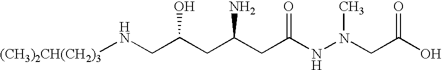

Step 1: t-Butyl {N′-[(3R)-t-butoxycarbonylamino-(5R)-t-butyldimethylsilanyloxy-6-[(4-methylpentyl)-(2-nitrobenzenesulfonyl)amino]hexanoyl]-N-methylhydrazino}acetate (18, R¹⁸=4-methylpentyl, R⁵=OTBDMS) was prepared from t-butyl {N′-[(3R)-t-butoxycarbonylamino-(5R)-t-butyldimethylsilanyloxy-6-(2-nitrobenzenesulfonylamino)hexanoyl]-N-methylhydrazino}acetate (17, R⁵=OTBDMS) and 4-methylpentanol following the Method X in 80% yield, after purifying by column chromatography using 45% ethyl acetate in hexanes as an eluent. R_{f}=0.25 (50% EtOAc/hexanes). ¹H NMR (300 MHz, CDCl₃) δ8.03 (m, 1H), 7.87 (s, 0.5H), 7.68 (m, 2H), 7.60 (m, 1H), 7.34 (s, 0.5H), 5.29 (m, 1H), 4.00-3.82 (m, 2H), 3.60-3.28 (m, 6H), 2.76 (s, 1.5H), 2.70 (s, 1.5H), 2.36 (m, 1H), 1.85 (m, 1H), 1.63 (m, 4H), 1.45 (s, 9H), 1.40 (s, 9H), 1.00 (m, 3H) 0.86 (s, 9H), 0.80 (d, 6H), 0.05 (s, 3H), 0.01 (s, 3H). MS (m/z): 788.3 [M+H]⁺.

Step 2: {N′-[(3R)-Amino-(5R)-hydroxy-6-(4-methylpentyllamino)hexanoyl]-N-methylhydrazino}acetic acid (19, R¹⁸=4-methylpentyl, R⁵=OH) was prepared from t-butyl {N′-[(3R)-t-butoxycarbonylamino-(5R)-t-butyldimethylsilanyloxy-6-[(4-methylpentyl)-(2-nitrobenzenesulfonyl)amino]hexanoyl]-N-methylhydrazino}acetate (18, R¹⁸=4-methylpentyl, R⁵=OTBDMS) in two steps following Method Y and Method T in 80% yield for two steps. ¹H NMR (300 MHz, D₂O) δ4.07 (m, 1H), 3.77 (m, 1H), 3.57 (m, 2H), 3.07 (m, 2H), 2.98 (m, 2H), 2.61 (s, 3H), 2.58 (m, 2H), 1.88-1.70 (m, 2H), 1.62 (m, 2H), 1.49 (m, 1H), 1.16 (m, 2H), 0.79 (d, 6H). ¹³C NMR (75 MHz, D₂O) δ63.8, 58.6, 52.3, 48.5, 46.1, 44.6, 36.1, 35.8, 35.1, 27.2, 23.6, 21.9 (2C). MS (m/z): 331.6 [M−H]⁻.

### Example 5

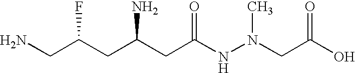

Step 1; t-Butyl (3S)-t-butoxycarbonylamino-4-hydroxybutyrate was synthesized according to the Method A in 85% yield from aspartic acid derivative 1 (R¹¹=R¹²=H), after purification by column chromatography using 30% ethyl acetate in hexanes as an eluent. R_{f}=0.66 (40% ethyl acetate in hexanes). ¹H NMR (CDCl₃) δ1.45 (s, 18H), 2.52 (m, 2H), 3.72 (d, J=5.1 Hz, 2H), 3.97 (m, 1H), 5.24 (bs, 1H), 9.67 (s, 1H). MS (m/z): 298 (M+Na).

Step 2: t-Butyl (3S)-t-butoxycarbonylamino-4-oxobutyrate (2, R¹¹=R¹²=H) was prepared according the Method B from t-butyl (3S)-t-butoxycarbonylamino-4-hydroxybutyrate in quantitative yield and used in the next step without further purification. R_{f}=0.66 (40% ethyl acetate in hexanes). ¹H NMR ¹H NMR (CDCl₃) δ1.44 (s, 9H), 1.46 (s, 9H), 2.71-2.95 (m, 2H), 4.34 (m, 1H), 5.62 (m, 1H), 9.67 (s, 1H).

Step 3: t-Butyl (3S)-t-butoxycarbonylamino-4-oxobutyrate (2, R¹¹=R¹²=H) was condensed with triethyl 2-fluoro-2-phosphonoacetate (12 mL, 60 mmol) according to the Method C to give (4S)-t-butoxycarbonylamino-2-fluorohex-2-enedioic acid 6-t-butyl ester 1-ethyl ester (3, R⁵=F, R¹¹=R¹²=H) in 88% yield, after purification by silica gel column chromatography using 30% ethyl acetate in hexanes as an eluent (1:9 mixture of E:Z isomers). R_{f}=0.54 (20% ethyl acetate in hexanes). ¹H NMR (CDCl₃) δ1.36 (t, J=7.2 Hz, 3H), 1.43 (s, 9H), 1.45 (s, 9H), 2.65 (m, 2H), 4.31 (q, J=7.2 Hz, 2H), 5.27 (m, 1H), 5.54 (bs, 1H), 6.04 (dd, J=9 Hz, J=20 Hz, 1H). MS (m/z): 384 (M+Na).

Step 4: (4R)-t-Butoxycarbonylamino-2-fluorohexanedioic acid 6-t-butyl ester 1-ethyl ester (4, R⁵=F, R¹¹=R¹²=H) was synthesized from (4S)-t-butoxycarbonylamino-2-fluorohex-2-enedioic acid 6-t-butyl ester 1-ethyl ester (3, R⁵=F, R¹¹=R¹²=H) in quantitative yield according to the Method D. R_{f}=0.47 (20% ethyl acetate in hexanes). ¹H NMR (CDCl₃) δ1.49 (m, 3H), 1.64 (s, 18H), 2.35 (t, J=6.8 Hz, 1H), 2.41 (t, 6.6 Hz, 1H), 2.71 (bs, 2H), 4.45 (m, 2H), 5.11 (m, 1H), 5.29 (m, 1H). MS (m/z): 386 (M+Na).

Step 5: t-Butyl (3R)-t-butoxycarbonylamino-(5R/S)-fluoro-6-hydroxyhexanate was synthesized from (4R)-t-butoxycarbonylamino-2-fluorohexanedioic acid 6-t-butyl ester 1-ethyl ester (4, R⁵=F, R¹¹=R¹²=H) in 80% yield as a diastereomeric mixture following the Method E, after purification by silica gel column chromatography using 50% ethyl acetate in hexanes as an eluent. R_{f}=0.25 (40% ethyl acetate in hexanes). ¹H NMR (CDCl₃) δ1.43 (s, 9H), 1.45 (s, 9H), 1.93 (m, 2H), 2.50 (t, J=6.0 Hz, 2H), 3.70-3.82 (m, 2H), 4.25 (bs, 1H), 4.59-4.87 (m, 1H), 5.17 (d, J=8.7 Hz, 1H). MS (m/z): 344 (M+Na).

Step 6: t-Butyl (3R)-t-butoxycarbonylamino-5-fluoro-6-methanesulfonyloxyhexanate was derived from t-butyl (3R)-t-butoxycarbonylamino-(5R/S)-fluoro-6-hydroxyhexanate and methanesulfonyl chloride according to the Method F in quantitative yield and used in the next step without purification. R_{f}: 0.38 (40% ethyl acetate in hexanes). MS (m/z): 422 (M+Na).

Step 7: t-Butyl 6-azido-(3R)-t-butoxycarbonylamino-(5R/S)-fluorohexanate was prepared, as a diastereomeric mixture, from t-butyl (3R)-t-butoxycarbonylamino-(5R/S)-fluoro-6-methanelsulfonyloxyhexanate in 91% yield according to Method G. The diastereomeric mixture was separated by silica gel column chromatography using 10% ethyl acetate in hexanes as an eluent. The higher R_{f} azidoester (Isomer A: t-butyl 6-azido-(3R)-t-butoxycarbonylamino-(5S)-fluorohexanate, R_{f}=0.55, 30% hexanes in ethyl acetate, 36%) was the undesired isomer and the lower R_{f} azidoester (Isomer B:: t-butyl 6-azido-(3R)-t-butoxycarbonylamino-(5R)-fluorohexanate R_{f}=0.44, , 30% hexanes in ethyl acetate, 36%) was the desired isomer. Isomer A: ¹H NMR (CDCl₃) δ1.43 (s, 9H), 1.45 (s, 9H), 1.81-2.08 (m, 2H), 2.48 (m, 2H), 3.44-3.54 (m, 2H), 4.02 (m, 1H), 4.65-4.86 (m, 1H), 5.17 (m, 1H). MS(m/z): 369 (M+Na). Isomer B: ¹H NMR (CDCl₃) δ1.43 (s, 9H), 1.45 (s, 9H), 1.93 (m, 2H), 2.52 (d, J=5.5 Hz, 2H), 3.36-3.45 (m, 2H), 4.10 (m, 1H), 4.68-4.87 (m, 1H), 5.15 (d, J=8.2 Hz, 1H). MS (m/z): 369 (M+Na).

Step 8: 6-Azido-(3R)-t-butoxycarbonylamino-(5R)-fluorohexanoic acid (R⁵=F, R¹¹=R¹²=H) was obtained from t-butyl 6-azido-(3R)-t-butoxycarbonylamino-(5R)-fluorohexanate according to the Method H in 93% yield. R_{f}=0.46 (ethyl acetate). ¹H NMR (CDCl₃) δ1.44 (s, 9H), 1.91-1.99 (m, 2H), 2.70 (m, 2H), 3.39-3.47 (m, 2H), 4.14 (m, 1H), 4.70-4.87 (m, 1H), 5.14 (m, 1H), 6.14 (m, 1H). MS (m/z): 289 (M−H).

Step 9: t-Butyl {N′-[6-azido-(3R)-t-butoxycarbonylamino-(5R)-fluorohexanoyl]-N-methylhydrazino}acetate (10 R²=R³=R⁴=R⁶=R⁷=R¹¹=R¹²=H, R⁵=F, R¹³=CH₃, X=N, R¹⁴=CH₂CO₂-t-Bu) was prepared from 6-Azido-3R-t-butoxycarbonylamino-5R-fluorohexanoic acid (R⁵=F, R¹¹=R¹²=H) and t-butyl (N-methylhydrazino)acetate following the Method P in 92% yield, after purification by column chromatography using ethyl acetate as an eluent. R_{f}=0.46 (ethyl acetate). ¹H NMR (300 MHz, CDCl₃) δ1.32 (s, 9H), 1.43 (s, 9H), 1.80-1.90 (m, 2H), 2.75 (br d, 2H), 2.64 (s, 3H), 3.30-3.40 (m, 2H), 3.50 (s, 2H), 4.11 (m, 1H), 4.60-4.85 (m, 1H), 5.32 (bs, 1H); MS (m/z): 455 (M+23).

Step 10: t-Butyl {N′-[6-amino-(3R)-t-butoxycarbonylamino-(5R)-fluorohexanoyl]-N-methylhydrazino}acetate (11, R²=R³=R⁴=R⁶=R⁷=R¹¹=R¹²=H, R⁵=F, R¹³=CH₃, X=N, R¹⁴=CH₂CO₂-t-Bu) was prepared from t-butyl {N′-[6-azido-(3R)-t-butoxycarbonylamino-(5R)-fluorohexanoyl]-N-methylhydrazino}acetate (10 R²=R³=R⁴=R⁶=R⁷=R¹¹=R¹²=H, R⁵=F, R¹³=CH₃, X=N, R¹⁴=CH₂CO₂-t-Bu) according to the Method R in 94% yield, after purification by column ion exchange column chromatography. ¹H NMR (300 MHz, CDCl₃) δ1.40 (s, 9H), 1.52 (s, 9H), 2.08 (m, 2H), 2.60 (br d, 2H), 2.91 & 2.94 (two s, 3H), 3.00-3.18 (m, 2H), 3.76 (s, 2H), 4.05 (m, 1H), 4.40-4.65 (m, 1H), 5.66 (br d, 1H), 7.56 & 8.08 (two s, 1H); MS (m/z): 429 (M+Na).

Step 11: {N′-[(3R)-Amino-6-amino-(5R)-fluorohexanoyl]-N-methylhydrazino}acetic acid (12, , R²=R³=R⁴=R⁶=R⁷=R¹¹=R¹²=H, R⁵=F, R¹³=CH₃, X=N, R¹⁴=CH₂CO₂-t-Bu) was prepared from t-butyl [N′-(6-amnio-3R-t-butoxycarbonylamino-5R-fluorohexanoyl)-N-methylhydrazino]acetate (11, R²=R³=R⁴=R⁶=R⁷=R¹¹=R¹²=H, R⁵=F, R¹³=CH₃, X=N, R¹⁴=CH₂CO₂H) using the Method T in 71% yield as a pale yellow solid. 1H NMR (300 MHz, CD₃OD) δ2.11-2.29 (m, 2H), 2.74 (m, 2H), 2.76 (s, 3H), 3.33-3.48 (m,2H), 3.74 (s, 2H), 3.98 (m, 1H), 5.07-5.50 (m, 1H); MS (m/z): 249 (M−1).

### Example 6

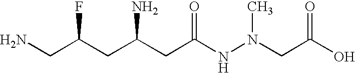

Step 1: 6-Azido-(3R)-t-butoxycarbonylamino-(5S)-fluorohexanoic acid (R⁵=F, R¹¹=R¹²=H) was obtained from t-butyl 6-azido-(3R)-t-butoxycarbonylamino-(5S)-fluorohexanate according to the Method H in 87% yield. R_{f}: 0.46 (ethyl acetate). R_{f}=0.45 (ethyl acetate). ¹H NMR (300 MHz, CDCl₃) δ1.44 (s, 9H), 1.89-2.10 (m, 2H), 2.65 (m, 2H), 3.26-3.44 (m, 2H), 4.08 (m, 1H), 4.66-4.88 (m, 1H), 5.13-5.15 (m, 1H). MS (m/z): 289 (M−1).

Step 2: t-Butyl {N′-[6-azido-(3R)-t-butoxycarbonylamino-(5S)-fluorohexanoyl]-N-methylhydrazino}acetate (10, R²=R³=R⁵=R⁶=R⁷=R¹¹=R¹²=H, R⁴=F, R¹³=CH₃, X=N, R¹⁴=CH₂CO₂-t-Bu) was prepared from 6-Azido-(3R)-t-butoxycarbonylamino-(5S)-fluorohexanoic acid (R⁵=F, R¹¹=R¹²=H) and t-butyl (N-methylhydrazino)acetate following the Method P in 49% yield, after purification by column chromatography using ethyl acetate as an eluent. R_{f}=0.73 (ethyl acetate). ¹H NMR (300 MHz, CDCl₃) δ1.43 (s, 9H), 1.48 (s, 9H), 1.80-1.99 (m, 2H), 2.31-2.38 (m, 2H), 2.75 (s, 3H), 3.40-3.54 (m, 2H), 3.56 (s, 2H), 4.04-4.08 (m, 1H), 4.60-4.83 (m, 1H), 5.45-5.60 (m, 1H), MS (m/z): 433 (M+H), 455 (M+Na).

Step 3: t-Butyl {N′-[6-amnio-(3R)-t-butoxycarbonylamino-(5S)-fluorohexanoyl]-N-methylhydrazino}acetate (11, R²=R³=R⁵=R⁶=R⁷=R¹¹=R¹²=H, R⁴=F, R¹³=CH₃, X=N, R¹⁴=CH₂CO₂-t-Bu) was prepared from t-Butyl {N′-[6-azido-(3R)-t-butoxycarbonylamino-(5S)-fluorohexanoyl]-N-methylhydrazino}acetate (10, R²=R³=R⁵=R⁶=R⁷=R¹¹=R¹²=H, R⁴=F, R¹³=CH₃, X=N, R¹⁴=CH₂CO₂-t-Bu) according to the Method R in 80% yield, after purification by ion exchange column chromatography. R_{f}=0.26 (Methanol). ¹H NMR (300 MHz, CDCl₃) δ1.42 (s, 9H), 1.47 (s, 9H), 2.39 (m, 2H), 2.72 (d, 3H), 2.87 (m, 2H), 3.40 (m, 2H), 3.56 (s, 2H), 4.02-4.11 (m, 1H), 4.42-4.76 (m, 1H), 5.40-5.60 (m, 1H). MS (m/z): 429 (M+Na).

Step 4: {N′-[(3R)-Amino-6-amino-(5S)-fluorohexanoyl]-N-methylhydrazino}acetic acid (12, R²=R³=R⁵=R⁶=R⁷=R¹¹=R¹²=H, R⁴=F, R¹³=CH_{3,} X=N, R¹⁴=CH₂CO₂H) was prepared from t-butyl {N′-[6-amnio-(3R)-t-butoxycarbonylamino-(5S)-fluorohexanoyl]-N-methylhydrazino}acetate (11 R²=R³=R⁵=R⁶=R⁷=R¹¹=R¹²=H, R⁴=F, R¹³=CH₃, X=N, R¹⁴=CH₂CO₂-t-Bu) using the Method T in 83% yield as a pale yellow solid. ¹H NMR (300 MHz, CDCl₃) δ2.06-2.25 (m, 2H), 2.69 (m, 2H), 2.73 (s, 3H), 3.31-3.45 (m, 2H), 3.70 (s, 2H), 3.96 (m, 1H), 5.08-5.38 (m,1H), MS (m/z): 251 (M+H).

### Example 7

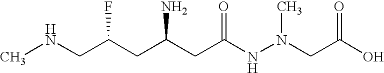

Step 1: t-Butyl {N′-[(3R)-t-butoxycarbonylamino-(5R)-fluoro-6-(2-nitrobenzenesulfonylamino)hexanoyl]-N-methylhadrazino}acetate (17, R⁵=F) was prepared from t-butyl {N′-[6-amino-(3R)-t-butoxycarbonylamino-(5R)-fluorohexanoyl]-N-methylhadrazino}acetate (11a, R⁵=F) according to the Method V in 68% yield, after purification by column chromatography using ethyl acetate as an eluent. R_{f}=0.53 (ethyl acetate). 1H NMR (300 MHz, CDCl₃) δ1.42 (s, 9H), 1.48 (s, 9H), 1.82-2.00 (m, 2H), 2.36-2.38 (m, 2H), 2.70 & 2.74 (two s, 3H), 3.33-3.50 (m, 2H), 3.55 (s, 2H), 4.01-4.07 (m, 1H), 4.62-4.79 (m, 1H), 5.48-5.51 (m, 1H), 5.75 (bs, 1H), 7.73-7.76 (m, 2H), 7.87-7.90 (m, 1H), 8.10-8.13 (m, 1H). MS (m/z): 614 (M+Na).

Step 2: t-Butyl (N′-{(3R)-t-butoxycarbonylamino-(5R)-fluoro-6-[methyl(2-nitrobenzenesulfonyl)amino]hexanoyl}-N-methylhadrazino)acetate (18, R⁵=F, R¹⁸=Me) was prepared from t-butyl {N′-[(3R)-t-butoxycarbonylamino-(5R)-fluoro-6-(2-nitrobenzenesulfonylamino)hexanoyl]-N-methylhadrazino}acetate (17, R⁵=F) and methyl alcohol according to Method X in 87% yield, after purification by column chromatography using 100% ethyl acetate as an eluent. R_{f} 0.73 (ethyl acetate). ¹H NMR (300 MHz, CDCl₃) δ1.43 (s, 9H), 1.48 (s, 9H), 1.85-1.99 (m, 2H), 2.39-2.40 (m, 2H), 2.71 & 2.75 (two s, 3H), 2.99 (s, 3H), 3.43-3.56 (m, 2H), 3.56 (s, 2H), 4.01-4.07 (m, 1H), 4.75-4.92 (m, 1H), 5.49-5.62 (m,2H), 7.73-7.76 (m, 2H), 7.87-7.90 (m, 1H), 8.10-8.13 (m, 1H). MS (m/z): 628 (M+Na).

Step 3: t-Butyl {N′-[3R)-t-butoxycarbonylamino-(5R)-fluorohexanoyl-6-methylamino]-N-methylhydrazino}acetate was prepared by the removal of the sulfonamide protecting group in t-butyl (N′-{(3R)-t-butoxycarbonylamino-(5R)-fluoro-6-[methyl(2-nitrobenzenesulfonyl)amino]hexanoyl}-N-methylhadrazino)acetate (18, R⁵=F, R¹⁸=Me) using the Method Y in 30% yield, after purifying by ion exchange column chromatography (Mega bound elut SCX column, sulfonic acid on silica gel). R_{f}=0.26 (5% methanol in ethyl acetate). ¹H NMR (300 MHz, CDCl₃) δ1.42 (s, 9H), 1.48 (s, 9H), 1.84-1.95 (m,2H), 2.39-2.41 (m, 2H), 2.45 (s, 3H), 2.71 & 2.75 (two s, 3H), 3.36-3.55 (m, 2H), 3.56 (s, 2H), 4.02-4.11 (m, 1H), 4.67-4.86 (m, 1H), 5.60 (m, 1H). MS (m/z): 421 (M+H).

Step 4: {N′-[(3R)-Amino-(5R)-fluoro-6-methylaminohexanoyl]-N-methylhydrazino}acetic acid (19, R¹⁸ =Me, R⁵=F) was prepared from t-butyl {N′-[(3R)-t-butoxycarbonylamino-(5R)-fluoro-6-methylaminohexanoyl]-N-methylhydrazino}acetate according to the Method T in 55% yield. ¹H NMR (300 MHz, D₂O) δ2.06-2.21 (m, 2H), 2.69-2.73 (m, 2H), 2.75 (s, 3H), 2.81 (s, 3H), 3.32-3.45 (m, 2H), 3.74 (s, 2H), 3.93-3.98 (m, 1H), 5.08-5.38 (m, 1H), MS (m/z): 263 (M−1).

### Example 8

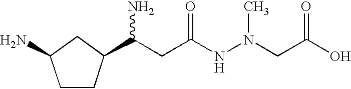

Step 1: [(3S)-Hydroxymethylcyclopentyl]-(1R)-carbamic acid t-butyl ester was prepared from (3R)-t-butoxycarbonylaminocyclopentane-(1S)-carboxylic acid in 75% yield following the Method A, after purification by column chromatography using 40% ethyl acetate in hexanes as an eluent. R_{f}=0.27 (40% ethyl acetate in hexanes). ¹H NMR (300 MHz, CDCl₃) δ1.33 (m, 1H), 1.64 (s, 12H), 1.94-2.04 (m, 1H), 2.05-2.21 (m, 1H), 2.38 (m, 2H), 3.77 (d, J=4.6 Hz, 2H), 4.14 (m, 1H). MS (m/z): 238 (M+Na).

Step 2: [(3S)-Formylcyclopentyl]-(1R)-carbamic acid t-butyl ester was synthesized [(3S)-Hydroxymethylcyclopentyl]-(1R)-carbamic acid t-butyl ester in quantitative yield according to the Method B and used in the next reaction without further purification. R_{f}: 0.53 (40% ethyl acetate in hexanes).

Step 3: To a stirred suspension of sodium hydride (0.2 g, 5.00 mmol) in anhydrous tetrahydrofuran (10 mL) at 0° C. was added t-butyl phosphonoacetate (1.44 g, 5.70 mmol) (10 mL). After completion of addition, the reaction mixture was stirred at room temperature for 30 minutes and then added a solution of [(3S)-formylcyclopentyl]-(1R)-carbamic acid t-butyl ester (0.925 g, 4.30 mmol) in tetrahydrofuran (10 mL) at 0° C. This was stirred at room temperature for 2 h and then the reaction mixture was acidified with hydrochloric acid (0.5 N, 10 mL). This was extracted with ethyl acetate (2×50 mL), the combined organic layer was washed with brine, dried (MgSO₄), and the solvent was removed in vacuo to give the crude t-butyl 3-[(3R)-t-butoxycarbonylamino-(1S)-cyclopentyl]acrylate, which was purified by silica gel column chromatography using 10% ethyl acetate in hexanes as an eluent (0.83 g, 63%). R_{f}=0.71 (20% ethyl acetate in hexanes). ¹H NMR (300 MHz, CDCl₃) δ1.37-1.48 (m, 2H), 1.64 (S, 9H), 1.65 (S, 9H), 2.03-2.20 (m, 2H), 2.22-2.42 (m, 2H), 2.81-2.87 (m, 1H), 4.21 (bs, H), 4.68 (bs, H), 5.92 (d, J=4.1 Hz, 1H), 7.02 (dd, J=4.1 Hz, 1H). MS (m/s): 334 (M+Na).

Step 4: To t-butyl 3-[(3R)-t-butoxycarbonylamino-(1S)-cyclopentyl]acrylate (0.3 g, 0.94 mmol) was added p-methoxybenzyl amine (1.0 mL, 7.00 mmol) and heated at 150° C. with stirring for 60 h. The excess benzyl amine was removed in vacuo. The reside obtained was adsorbed on silica gel and purified by silica gel column chromatography using 25% ethyl acetate in hexanes as an eluent to give t-butyl (3R/S)-[(3R)-t-butoxycarbonylamino-(1S)-cyclopentyl]-3-(4-methoxybenzylamino)propionate (0.24 g, 65%). R_{f}=0.29 (40% ethyl acetate in hexanes). ¹H NMR (300 MHz, CDCl₃) δ1.32 (m, 2H), 1.63 (s, 9H), 1.65 (S, 9H), 1.78-2.07 (m, 4H), 2.28-2.34 (m, 2H), 2.57-2.59 (m, 2H), 3.05 (m, 1H), 3.88 (m,2H), 3.99 (s, 3H), 7.03 (d, J=8.7 Hz, 2H), 7.43 (d, J=8.7 Hz, 2H). MS (m/z): 449 (M+H)

Step 5: To a solution of t-butyl (3R/S)-[(3R)-t-butoxycarbonylamino-(1S)-cyclopentyl]-3-(4-methoxybenzylamino)propionate (0.150 g, 0.33 mmol) in ethyl acetate (10 mL) was added Pd/C (50 mg, 10%) and di-t-butyl dicarbonate (0.400 g, 1.33 mmol) and the resultant reaction mixture was hydrogenated at room temperature using a balloon full of hydrogen for 16 h. The catalyst was filtered through a pad of celite and washed with ethyl acetate. The combined filtrate was concentrated in vacuo to give acylated product (0.150 g, 82%), but without the removal of the N-4-methoxybenzyl group. This was purified on silica gel column chromatography using 30% ethyl acetate in hexanes as an eluent to obtain t-butyl (3R/S)-[(3R)-t-butoxycarbonylamino-(1S)-cyclopentyl)]-3-[t-butoxycarbonyl-(4-methoxybenzyl)amino]propionate (0.12 g, 65%). R_{f}=0.53 (40% ethyl acetate in hexanes). ¹H NMR (300 MHz, CDCl₃) δ0.81 (m, 2H), 1.35 (s, 18H), 1.36 (s, 9H), 1.60-2.22 (m, 4H), 2.19-2.43 (m, 4H), 2.57-2.59 (m, 2H), 3.73 (s, 3H), 4.06 (m, 1H), 5.03 (m, 1H), 6.78 (m, 2H), 7.19 (m, 2H). MS (m/z): 572 (M+Na).

Step 6: To a solution of t-butyl (3R/S)-[(3R)-t-butoxycarbonylamino-(1S)-cyclopentyl)]-3-[t-butoxycarbonyl-(4-methoxybenzyl)amino]propionate (0.150 g, 0.27 mmol) in a mixture of acetonitrile (5 mL) and water (1 mL), ceric ammonium nitrate (0.120 g, 0.32 mmol) was added. The reaction mixture was stirred overnight at room temperature and then removed the solvent in vacuo. The residue was extracted with ethyl acetate (50 mL), washed with water (20 mL) and dried (NaSO₄). Removal of solvent in vacuo resulted in crude product, which was purified by silica gel column chromatography using 50% ethyl acetate in hexanes as an eluent to give t-butyl (3R/S)-t-butyoxycarbonylamino-3-[(3R)-t-butoxycarbonylamino-(1S)-cyclopentyl]propionate (0.075 g, 65%), R_{f}=0.30 (20% ethyl acetate in hexanes). ¹H NMR (300 MHz, CDCl₃) δ1.19-1.36 (m, 2H), 1.63 (s, 9H), 1.64 (S, 18H), 2.01-2.48 (m, 5H), 2.53-2.70 (m, 2H), 3.99 (m, 1H), 4.72 (m, 1H), 4.93 (m, 1H), 5.27 (m, H). MS (m/z): 451 (M+Na).

Step 7: (3R/S)-t-butyoxycarbonylamino-3-[(3R)-t-butoxycarbonylamino-(1S)-cyclopentyl]propionic acid (14, , R²=R⁴=R⁵=R⁷=R¹¹=R¹²=H, R³ & R⁶ together forms a cyclopentyl ring) was synthesized from t-butyl (3R/S)-t-butyoxycarbonylamino-3-[(3R)-t-butoxycarbonylamino-(1S)-cyclopentyl]propionate according to the Method H in 99% yield and used in the next step without further purification. R_{f}=0.50 (ethyl acetate). ¹H NMR (300 MHz, CDCl₃) δ1.23-1.36 (m, 2H), 1.37 (s, 18H), 1.68-2.24 (m, 5H), 2.51 (bs, 2H), 4.12 (m, 1H), 5.09 (bs, H). MS (m/z): 371 (M−1).

Step 8: t-Butyl (N′-{(3R/S)-t-butoxycarbonylamino-3-[(3R)-t-butoxycarbonylamino-(1S)-cyclopentyl)]propionyl}-N-methylhydraino)acetate (15, R²=R⁴=R⁵=R⁷=R¹¹=R¹²=H, R³ & R⁶ together forms a cyclopentyl ring, R¹³=CH₃, X=N, R¹⁴=CH₂CO₂-t-Bu) was synthesized according to the Method P from (3R/S)-t-butyoxycarbonylamino-3R-[(3R)-t-butoxycarbonylamino-(1S)-cyclopentyl]propionic (14, , R²=R⁴=R⁵=R⁷=R¹¹=R¹²=H, R³ & R⁶ together forms a cyclopentyl ring) and t-butyl (N-methylhydrazino)acetate in 45% yield, after purification by column chromatography using ethyl acetate as an eluent. R_{f}=0.55 (ethyl acetate). ¹H NMR (300 MHz, CDCl₃) δ1.61 (S, 9H), 1.63 (S, 18H), 2.14-2.72 (m, 6H), 2.91 (S, 5H), 3.50-3.71 (m, 2H), 3.74 (s, 2H), 4.04 (m, 1H). MS (m/z): 516 (M+H).

Step 9: (N′-{(3R/S)-Amino-3-[(3R)-amino-(1S)-cyclopentyl]propionyl}-N-methylhydraino)acetic acid (16, R²=R⁴=R⁵=R⁷=R¹¹=R¹²=H, R³ & R⁶ together forms a cyclopentyl ring, R¹³=CH₃, X=N, R¹⁴=CH₂CO₂H) was prepared from the t-Butyl (N′-{(3R/S)-t-butoxycarbonylamino-3-[(3R)-t-butoxycarbonylamino-(1S)-cyclopentyl)]propionyl}-N-methylhydraino)acetate (15, R²=R⁴=R⁵=R⁷=R¹¹=R¹²=H, R³ & R⁶ together forms a cyclopentyl ring, R¹³=CH₃, X=N, R¹⁴=CH₂CO₂-t-Bu) according to the Method T. This was purified by ion-exchange column chromatography (Amberlite CG-50) to give the final product as ammonium salt in 86% yield . ¹H NMR (300 MHz, CDCl₃) δ1.31-1.43 (m, 2H), 1.55-1.74 (m, 2H), 1.84-1.87 (m, 2H), 2.04-2.28 (m, 2H), 2.41 (s, 2H), 244 (s, 3H), 2.91-2.96 (m, 1H), 3.20 (s, 2H), 3.50 (m, 1H). MS (m/z): 257 (M−H).

### Example 9

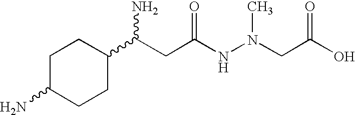

Step 1: (3R/S)-(4-Acetylaminophenyl)-3-aminopropionic acid (22, A=NHAc, B=H, a=b=c=C) was synthesized from N-(4-formylphenyl)acetamide according to the Method Z in quantitative yield. ¹H NMR (300 MHz, CD₃OD) δ2.33 (s, 3H), 3.26 (m, 2H), 4.89 (m, 1H), 7.60-8.00 (m, 4H). MS (m/z): 221(M−H).

Step 2: (3R/S)-(4-Acetylaminophenyl)-3-aminopropionic acid (22, A=NHAc, B=H, a=b=c=C, 3 g, 13.5 mmol) in hydrochloric acid (30 mL, 6 N) was heated to reflux for 20 hours with stirring. The solvent was removed in vacuo to provide amine hydrochloride of (3R/S)-amino-3-(4-aminophenyl)propionic acid (23, A=NH₂, B=H, a=b=c=C) as a yellow solid (0.9 g, 37%). ¹H NMR (300 MHz, D₂O) δ3.26 (m, 2H), 4.85 (m, 1H), 7.68 (m, 4H). MS (m/z): 179(M−H).

Step 3: (3R/S)-t-Butoxycarbonylamino-3-(4-t-butoxycarbonylaminophenyl)propionic acid (24, A=NHBoc, B=H, a=b=c=C) was synthesized (3R/S)-amino-(3R/S)-(4-aminophenyl)propionic acid (23, A=NH₂, B=H, a=b=c=C) in 0.5% yield according to Method AA, after purification by silica gel column chromatography using ethyl acetate as an eluent. R_{f}=0.4 (ethyl acetate). 1H NMR (300 MHz, DMSO-d₆) δ1.41 (s, 18H), 2.60 (m, 2H), 4.87 (m, 1H), 7.14 (d, J=8.5 Hz, 2H), 7.25 (d, J=8.5 Hz, 2H). MS (m/z): 379(M−1).

Step 4: t-Butyl {N′-[(3R/S)-t-butoxycarbonylamino-3-(4-t-butoxycarbonylaminophenyl)propionyl]-N-methylhydrazino}acetate (25, A=NHBoc, B=H, a=b=c=C) was prepared from (3R/S)-t-butoxycarbonylamino-3-(4-t-butoxycarbonylaminophenyl)propionic acid (24, A=NHBoc, B=H, a=b=c=C) and (N-methylhydrazino)acetate using Method P in 24% yield, after purification by silica gel column chromatography using 50% ethyl acetate in hexanes as an eluent. R_{f}=0.7 (ethyl acetate). ¹H NMR (300 MHz, CDCl₃) δ1.40 (s, 9H), 1.44 (s, 9H), 1.50 (s, 9H), 2.58 (m, 5H), 3.42 (m, 2H), 5.00 (m, 1H), 7.27 (m, 4H). MS (m/z): 523(M+H).

Step 5: {N′-[(3R/S)-Amino-3-(4-aminophenyl)propionyl]-N-methylhydrazino}acetic acid (26, A=NH₂, B=H, a=b=c=C) was synthesized according to Method T from the t-Butyl {N′-[(3R/S)-t-butoxycarbonylamino-3-(4-t-butoxycarbonylaminophenyl)propionyl]-N-methylhydrazino}acetate (25, A=NHBoc, B=H, a=b=c=C) in 99% yield. ¹H NMR (300 MHz, D₂O) δ2.52 (s, 3H), 3.00 (m, 2H), 3.52 (d, J=2.2 Hz, 2H), 4.91 (m, 1H), 7.54 (d, J=8.8 Hz, 2H), 7.65 (d, J=8.8 Hz, 2H). MS (m/z): 265(M−1).

Step 6: {N′-[(3R/S)-Amino-3-{(4R/S)-amino-(1R/S)-cyclohexyl}propionyl]-N-methylhydrazino}acetic acid (27, A=NH₂, B=H, a=b=c=C) was synthesized from {N′-[(3R/S)-Amino-3-(4-aminophenyl)propionyl]-N-methylhydrazino}acetic acid (26, A=NH₂, B=H, a=b=c=C) using the Method BB in 43% yield, after purification by ion exchange column chromatography. ¹H NMR (300 MHz, D₂O) δ1.20-3.65 (m, 12H), 2.52 (s, 3H), 3.44 (s, 2H), 4.90 (m, 1H). MS (m/z): 271(M−1).

### Example 10

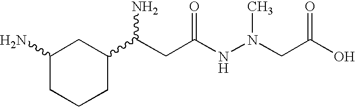

Step 1: (3R/S)-Amino-3-(3-nitrophenyl)proponic acid (22, A=H, B NO₂, a=b=c=C) was synthesized from 3-nitrobenzaldehyde (20, A=H, B =NO₂, a=b=c=C) according to the Method Z in 70% yield. ¹H NMR (300 MHz, CD₃OD) δ3.30 (m, 2H), 4.91 (m, 1H), 8.10 (m, 4H). MS (m/z): 209(M−1).

Step 2: To a solution of (3R/S)-amino-3-(3-nitrophenyl)proponic acid (22, A=H, B=NO₂, a=b=c=C, 5 g, 57 mmol) in methanol (70 mL) was added 10% Pd-C (0.7 g) and hydrogenated at 50 psi for 24 h. The catalyst was filtered off and the filtrate was concentrated to give the (3R/S)-amino-3-(3-aminophenyl)propionic acid (23, A=H, B=NH₂, a=b=c=C, 6.8 g, 65%). ¹H NMR (300 MHz, D₂O) δ3.40 (m, 2H), 4.91 (m, 1H), 7.30 (m, 4H).

Step 3: (3R/S)-t-Butoxycarbonylamino-3-(3-t-butoxycarbonylaminophenyl)propionic acid (24, A=H, B=NH₂, a=b=c=C) was synthesized from (3R/S)-amino-(3R/S)-(3-aminophenyl)propionic acid (23, A=H, B=NHBoc, a=b=c=C) in 3% yield using sodium hydroxide as a base according to Method AA, after purification by silica gel column chromatography using ethyl acetate as an eluent. R_{f}=0.4 (ethyl acetate). ¹H NMR (300 MHz, CD₃OD) δ3.00 (m, 2H), 5.00 (m, 1H), 7.31 (m, 4H). MS (m/z): 379(M−1).

Step 4: t-Butyl {N′-[(3R/S)-butoxycarbonylamino-3-(3-t-butoxycarbonylaminophenyl)propionyl]-N-methylhydrazino}acetate (25, A=H, B =NHBoc, a=b=c=C) was prepared from (3R/S)-t-butoxycarbonylamino-3-(3-t-butoxycarbonylaminophenyl)propionic acid (24, A=H, B=NH₂, a=b=c=C) and (N-methylhydrazino)acetate using Method P in 24% yield, after purification by silica gel column chromatography using 50% ethyl acetate in hexanes as an eluent. R_{f}=0.8 (ethyl acetate). ¹H NMR (300 MHz, CDCl₃) δ1.50 (m, 27H), 2.61 (m, 5H), 3.40 (m, 2H), 5.00 (m, 1H), 7.32 (m, 4H). MS (m/z): 523(M+H).

Step 5: {N′-[(3R/S)-Amino-3-(3-aminophenyl)propionyl]-N-methylhydrazino}acetic acid (26, A=H, B=NH₂, a=b=c=C) was synthesized according to Method T from t-butyl {N′-[(3R/S)-butoxycarbonylamino-3-(3-t-butoxycarbonylaminophenyl)propionyl]-N-methylhydrazino}acetate (25, A=H, B=NHBoc, a=b=c=C) in quantitative yield. ¹H NMR (300 MHz, D₂O) δ2.52 (s, 3H), 3.00 (m, 2H), 3.51 (d, J=3.3 Hz, 2H), 4.90 (m, 1H), 7.60 (m, 4H). MS (m/z): 265(M−1).

Step 6: {N′-[(3R/S)-Amino-3-{(3R/S)-amino-(1R/S)-cyclohexyl}propionyl]-N-methylhydrazino}acetic acid (27, A=H, B=NH₂, a=b=c=C) was synthesized from {N′-[(3R/S)-Amino-3-(3-aminophenyl)propionyl]-N-methylhydrazino}acetic acid (26, A=H, B=NH₂, a =b=c=C) using the Method BB in 59% yield, after purification by ion exchange column chromatography. ¹H NMR (300 MHz, D₂O) δ1.20-3.65 (m, 12H), 2.72 (s, 3H), 3.44 (s, 2H), 4.90 (m, 1H). MS (m/z): 271(M−1).

### Example 11

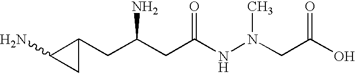

Step 1: t-Butyl (3R)-t-butoxycarbonylaminohex-5-enoate was prepared from N-t-butyloxycarbonyl-D-allylglycine in 66% yield according to the Method I, after purification by column chromatography using 9:1 hexanes/ethyl acetate mixture as an eluent. R_{f}=0.64 (50% EtOAc/hexanes). ¹H NMR (300 MHz, CDCl₃) δ1.42 (s, 18H), 2.30 (m, 2H), 2.42 (d, 1H), 2.58 (d, 1H), 3.95 (m, 1H), 5.08-5.15 (m, 2H), 5.75 (m, 1H). MS (m/z): 308 (M+Na).

Step 2: To a t-butyl (3R)-t-butyloxycarbonylaminohex-5-enoate (2.45 g, 8.59 mmol) in anhydrous dichloromethane (50 ml) at room temperature under nitrogen atmosphere was treated with rhodium (II) acetate dimer (Rh₂(OAc)₄, 76 mg, 0.17 mmol). A solution of ethyl diazoacetate (1.22 g, 10.7 mmol) in anhydrous DCM (50 ml) was added via syringe pump over 72 h. The catalyst was filtered off and the filtrate was concentrated in vacuo. The residue was purified by flash chromatography on silica gel to afford isomeric mixtures of (2R/S)-[3-t-butoxycarbonyl-(2R)-t-butoxycarbonylaminopropyl]cyclopropane-(1R/S)-carboxylic acid ethyl ester (0.978 g, 31%). R_{f}=0.45 (30% Ethyl acetate (EtOAc)/hexanes). ¹H NMR (300 MHz, CDCl₃) δ0.75-1.15 (m, 2H), 1.15-1.20 (m, 1H), 1.25 (m, 3H), 1.20 (s, 9H), 1.25 (s, 9H), 1.65-1-80 (m, 3H), 2.45 (m,2H), 3.89 (m, 1H), 4.10 (m, 2H). MS (m/z): 394 (M+Na).

Step 3: The diastereomeric mixture of (2R/S)-[3-t-butoxycarbonyl-(2R)-t-butoxycarbonylaminopropyl]cyclopropane-(1R/S)-carboxylic acid was prepared from (2R/S)-[3-t-butoxycarbonyl-(2R)-t-butoxycarbonylaminopropyl]cyclopropane-(1R/S)-carboxylic acid ethyl ester in 80% yield according Method J. ¹H NMR (300 MHz, CDCl₃) δ0.80-1.60 (m, 2H), 1.42 (s, 9H), 1.43 (s, 9H), 1.75 (m, 1H), 1.95 (m, 1H), 2.45 (m, 2H), 2.65 (m, 2H), 3.95 (m, 1H). MS (m/z): 342 (M−H)

Step 4: A solution of (2R/S)-[3-t-butoxycarbonyl-(2R)-t-butoxycarbonylaminopropyl]cyclopropane-(1R/S)-carboxylic acid (0.33 g, 0.96 mmol) in anhydrous t-butanol (15 ml) was treated with triethylamine (TEA) 0.28 ml, 2.02 mmol) and diphenylphosphoryl azide (0.24 ml, 1.06 mmol). The reaction mixture was heated at reflux overnight and then removed t-butanol. The residue was purified by flash chromatography to give isomeric mixtures of t-butyl (3R)-t-butoxycarbonylamino-4-[(2R/S)-t-butoxycarbonylamino-(1R/S)-cyclopropyl]butyrate (260 mg, 66%). R_{f}=0.37 (30% EtOAc/hexanes). ¹H NMR (300 MHz, CDCl₃) δ0.50-1.15 (m, 2H), 1.15 (m, 1H), 1.45 (s, 27H), 2.20-2.30 (m, 1H), 2.50 (m, 2H) 2.75 (m, 2H), 4.00 (m, 1H). MS (m/z): 437 (M+Na)

Step 5: t-butyl (3R)-t-butoxycarbonylamino-4-[(2R/S)-t-butoxycarbonylamino-(1R/S)-cyclopropyl]butyrate was hydrolyzed to give the (3R)-t-butoxycarbonylamino-4-[(2R/S)-t-butoxycarbonylamino-(1R/S)-cyclopropyl]butyric acid following Method H, using potassium hydroxide instead of sodium hydroxide, in 97% yield. ¹H NMR (300 MHz, CDCl₃) δ0.50-1.15 (m, 2H), 1.15 (m, 1H), 1.45 (s, 18H), 1.80-2.20 (m, 2H), 2.35 (m, 1H), 2.65 (m, 2H), 4.10 (m, 1H). MS (m/z): 357 (M−H)

Step 6: t-Butyl {N′-[(3R)-t-butoxycarbonylamino-4-((2R/S)-t-butoxycarbonylamio-(1R/S)-cyclopropyl)butyryl]-N-methylhydrazino}acetate (15, R³=R⁵=R⁶=R⁷=R¹¹=R¹²=H, R² & R⁴ together forms a cyclopropyl ring, R¹³=CH₃, X=N, R¹⁴=CH₂CO₂-t-Bu) was prepared from (3R)-t-butoxycarbonylamino-4-[(2R/S)-t-butoxycarbonylamino-(1R/S)-cyclopropyl]butyric acid and the t-butyl (N-methylhydrazino)acetate according to Method P in 70% yield, after purification by silica gel column chromatography. R_{f}=0.24 (Ethyl acetate). ¹H NMR (300 MHz, CDCl₃) δ0.50-1.00 (m, 2H), 1.15 (m, 1H), 1.40-1.55 (s, 27H), 2.30 (m, 1H), 2.35-2.60 (m, 2H), 2.75-80 (m, 4H), 2.55 (d, 3H), 4.10 (m, 1H). MS (m/z): 523 (M+Na)

Step 7: t-Butyl {N′-[(3R)-t-butoxycarbonylamino-4-((2R/S)-t-butoxycarbonylamino-(1R/S)-cyclopropyl)butyryl]-N-methylhydrazino}acetate (15, R³=R⁵=R⁶=R⁷=R¹¹R¹²=H, R² & R⁴ together forms a cyclopropyl ring, R¹³=CH₃, X=N, R¹⁴=CH₂CO₂-t-Bu, 70 mg, 0.14 mmol) was subjected to removal of the protecting groups according to the Method T which gave the {N′-[(3R)-amino-4-((2R/S)-amino-(1R/S)-cyclopropyl)butyryl]-N-methylhydrazino}acetic acid (16, R³=R⁵=R⁶=R⁷=R¹¹=R¹²=H, R² & R⁴ together forms a cyclopropyl ring, R¹³=CH₃, X=N, R¹⁴=CH₂CO₂H) in 98% yield. ¹H NMR (300 MHz, D₂O) δ0.65-1.10 (m, 2H), 1.35 (m, 1H), 1.70(m, 1H), 2.35-2.45 (m, 2H), 2.45-2.65 (m, 2H), 2.55(s, 3H), 3.55 (s, 2H), 3.90 (m, 1H). MS (m/z): 243 (M−H).

### Example 12

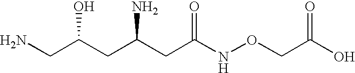

Step 1: To a stirred suspension of sodium hydride (4.0 g of 60% dispersion, 100 mmol), prewashed with hexanes, in N,N-dimethylformamide (300 mL) at 0° C. was added N-hydroxyphthalimide (16.3 g, 100 mmol) in several portions over 15 min period. After completion of the addition, the reaction mixture was stirred at room temperature for 20 min and then added t-butyl bromoacetate (19.5 g, 110 mmol) in one lot. The resultant reaction mixture was stirred at room temperature for 30 min and then decomposed by adding on to crushed ice (100 g). The precipitated solid was filtered, washed with water (50 mL), and dried under high vacuum to give t-butyl (1,3-dioxo-1,3-dihydroisoindol-2-yloxy)acetate (27 g, 97%). R_{f}=0.34 (1:1 Ethyl acetate/Hexanes, silica gel). ¹H NMR (300 MHz, CDCl₃) δ1.49 (s, 9H), 4.71 (s, 2H), 7.74-7.87 (m, 4H).

Step 2: To a stirred suspension of t-butyl (1,3-dioxo-1,3-dihydroisoindol-2-yloxy)acetate (6 g, 21.63 mmol) in ethanol was added hydrazine (6 mL) which resulted in exothermic reaction and a clear solution. Let the reaction mixture sit at room temperature for 2 h during which period a solid precipitated. The reaction mixture was diluted with water (200 mL) and this was extracted with ether (3×75 mL). The combined organic layer was washed with sodium bicarbonate solution (5%, 3×100 mL), water (3×100 mL), dried the organic layer (MgSO₄), and then removal of solvent gave the t-butyl 2-aminooxyacetate (1.1 g, 35%). ¹H NMR (300 MHz, CDCl₃) δ1.49 (s, 9H), 4.13 (s, 2H), 5.86 (bs, 2H).

Step 3: t-Butyl [6-azido-(3R)-t-butoxycarbonylamino-(5R)-(t-butyldimethylsilanyloxy)hexonylaminooxy]acetate (R²=R³=R⁴=R⁶=R⁷=R⁶=R⁷=R¹¹=R¹²=H, R⁵=OTBDMS, X=O, R¹⁴=CH₂CO₂-t-Bu) was prepared from the pentafluorophenyl [6-azido-(3R)-t-butoxycarbonylamino-(5R)-t-butyldimethylsilanyloxy]hexanate (R²=R³=R⁴=R⁶=R⁷=R⁶=R⁷=R¹¹=R¹²=H, R⁵=OTBDMS) and the t-butyl 2-aminooxyacetate according to the Method Q in 88% yield, after purification by column chromatography using a mixture of ethyl acetate and hexanes (4:6) as an eluent. R_{f}=0.30 (1:1 hexanes/ethyl acetate, silica gel). ¹H NMR (300 MHz, CDCl₃) δ0.01 (s, 6H), 0.92 (s, 9H), 1.49 (s, 9H), 1.62 (s, 9H), 1.78 (m, 2H), 2.05 (bm, 2H), 3.14-3.37 (ABq, J=12.9 Hz, 4.8 Hz, 2H), 3.94 (m, 2H), 4.34 (bs, 2H), 5.18 (bs, 1H), 9.12 (bs, 1H). MS (m/z): 554.3 (M+Na).

Step 4: t-Butyl [6-amino-(3R)-t-butoxycarbonylamino-(5R)-(t-butyldimethylsilanyloxy)hexonylaminooxy]acetate (R²=R³=R⁴=R⁶=R⁷=R⁶=R⁷=R¹¹=R¹²=H, R⁵=OTBDMS, X=O, R¹⁴=CH₂CO₂-t-Bu) was prepared from t-Butyl [6-azido-(3R)-t-butoxycarbonylamino-(5R)-(t-butyldimetjylsilanyloxy)hexonylaminooxy]acetate (R²=R³=R⁴=R⁶=R⁷=R⁶=R⁷=R¹¹=R¹²=H, R⁵=OTBDMS, X=O, R¹⁴=CH₂CO₂-t-Bu) according to the Method S in 84% yield, after purification by ion exchange column chromatography. ¹H NMR (300 MHz, CDCl₃) δ0.08 (s, 3H), 0.1 (s, 3H), 0.9 (s, 9H), 1.47 (s, 9H), 1.5 (s, 9H), 1.78 (m, 2H), 2.41 (m, 2H,), 2.6-2.8 (m, 2H), 3.8 (m, 1H, 3.91 (m, 1H), 4.13 (s, 2H0, 5.46 (bs, 1H). MS (m/z): 506.7 (M+H), 528.7 (M+Na).

Step 5: [(3R)-Amino-6-amino-(5R)-hydroxyhexonylaminooxy]acetic acid (R²=R³R⁴=R⁶=R⁷=R⁶=R⁷=R¹¹=R¹²=H, R⁵=H, X=O, R¹⁴=CH₂CO₂H) was prepared from the t-butyl [6-amino-(3R)-t-butoxycarbonylamino-(5R)-(t-butyldimetjylsilanyloxy)hexonylaminooxy](R²=R³=R⁴=R⁶=R⁷=R⁶=R⁷=R¹¹=R¹²=H, R⁵=OTBDMS, X=O, R¹⁴=CH₂CO₂-t-Bu) according to the Method U in 67% yield, after purification by ion exchange column chromatography. ¹H NMR (300 MHz, CDCl₃) δ1.80 (m, 2H), 2.53 (m, 2H), 2.77-3.15 (m, 2H), 3.37-3.71 (m, 2H), 4.01 (s, 2H). MS (m/z): 234 (M−1).

B. In Vitro Testing

The ability of a compound to treat bacterial infection was measured by in vitro assay described below. The compounds were kept in DMSO at 10 mg/mil and diluted into the test medium for the assay.

The strains used for the assay include the following:

Escherichia coli VECO1003

Escherichia coli VECO2096

Escherichia coli VECO2526

Klebsiella pneumoniae VKPN1001

Enterobacter cloacae VECL1001

Pseudomonas aeruginosa VPAE1003

Staphylococcus aureus VSAU1003

Streptococcus pneumoniae VSPN1005

Helicobacter pylori HPY1001

### (1) Liquid medium, Nutrient Broth and Mueller-Hinton Broth with 50% Human Serum

Preparation of Plates with Two-fold Dilutions of Test Compound

Compounds were suspended in a DMSO solution containing 10 mg/ml of compound. The appropriate amount of compound was added to a well of the first column of a 96-well microtiter plate containing 100 μl of Nutrient Broth (NB) or Mueller-Hinton Broth with 50% human serum (MHB+HS), for S. pneumoniae the NB medium was supplemented with 3 or 5% lysed horse blood. The rest of the wells in the row had 50 μl of the same medium in each well. After adding the compound to the first well and mixing thoroughly, 50 μl was taken out, and mixed to the second well of the row. This operation was repeated until the well of column 11 was reached and, after mixing, 50 μl were discarded. The last column was a control well that does not contain test compound. A concentration gradient of two-fold dilution was achieved by this method. A distinct compound was added to the first column well of the next row of the microtiter plate, and the procedure was repeated until all the rows are used. The plate was then ready for inoculation.

### Preparation of Inoculum

Bacterial strains were inoculated from a frozen stock on Mueller-Hinton Agar (Difco) or Blood Agar for S. pneumoniae and grown at 35° C. overnight. Cells were suspended in sterile saline to 0.5 McFarland standard. The inoculum was diluted approximately 1:1000 in the appropriate medium to reach an inoculum size of 105 cfu/ml (NB, NB supplemented with 3 or 5% lysed horse blood for S. pneumoniae, or MHB+HS). The diluted cell suspension was used to inoculate the microtiter plate containing two-fold dilution of the test compound. Each well of the microtiter plate received 50 μl of the cell suspension. The final volume was 100 μl (50 μl from the inoculum and 50 μl of the medium containing the compound).

### Incubation of the Plates

The plates were incubated at 35 C. for 16 to 20 hours, the minimal inhibitory concentration (MIC) was recorded and was defined as the lowest concentration of compound where there is no visible growth.

### (2) Solid Medium, Mueller-Hinton Agar with 50% Human Serum Preparation of Petri Dishes for Assessing MIC in Solid Medium

Compounds were suspended in a DMSO solution containing 10 mg/ml of compound. The appropriate amount of compound was added to melted Mueller-Hinton Agar with 50% human serum (MHA+HS) at 50° C. The medium has been prepared by adding human serum to a melted Mueller-Hinton Agar (MHA) at two fold the concentration. MHA+HS has 50% human serum and normal strength of MHA. The appropriate concentration of compound was added to 4 ml of melted MHA+HS, and poured in a 6 cm diameter petri dish, and the agar was allowed to solidify at room temperature. A set of petri dishes, each dish containing half the concentration of compound of the previously poured dish, was made and used to assess the MIC in solid medium

### Preparation of Inoculum

Bacterial strains were inoculated from a frozen stock on Mueller-Hinton Agar (Difco) or Blood Agar for S. pneumoniae and grown at 35 C. overnight. Cells are suspended in sterile saline to 0.5 McFarland standard. The inoculum was diluted approximately 1:10 in sterile saline water to obtain a bacterial suspension at 107 cfu/ml. This cell suspension was used to inoculate the petri dishes with varying concentration of test compound by applying 2 μl on the surface (approximately 104 cfu).

### Incubation of Plates

Petri dishes were incubated at 35 C. for 16 to 20 hours, the minimal inhibitory concentration (MIC) was recorded and was defined as the lowest concentration of compound where there is no visible growth.

### In Vitro Data

Each of the preferred compounds described in synthetic examples 1-12 above exhibited activity (i.e., MIC<128 μg/ml) against a variety of tested bacterial strains.

C. In Vivo Testing

Escherichia coli ATCC 25922 was used to test the efficacy of compounds in a mouse septicemia model using six ICR male mice per group. Mice were inoculated intraperitoneally with 2×10⁵ cfu/mouse in 0.5 ml of Brain-Heart Infusion broth supplemented with 5% mucin. The compounds were tested at 40, 20, 10, 5 and 2.5 mg/kg orally (PO) or at 20, 10, 5 and 2.5 mg/kg intravenously (IV) at 1 and 5 hours after bacterial inoculation. Controls included vehicle control (0.9% NaCl, 10 ml/kg) and an antibiotic control with ampicillin. Mortality was recorded for 7 days, and an ED₅₀ was determined by non-linear regression.

### In Vivo Data

The following compounds exhibited biological activity (i.e., ED50 <40 mg/kg) when tested: 19 (R¹⁸=Me, R⁵=OH); 12 (R²=R³=R⁴=R⁶=R⁷=R¹¹=R¹²=H, R⁵=F, R¹³=CH₃, X=N, R¹⁴=CH₂CO₂t-Bu); and 19 (R¹⁸=Me, R⁵=F).

From the foregoing description, various modifications and changes in the composition and method will occur to those skilled in the art. All such modifications coming within the scope of the appended claims are intended to be included therein.

What is claimed is:

1. A compound of formula (I): 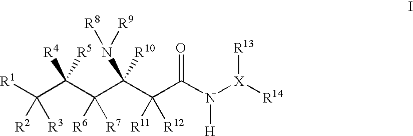
wherein the substituents are as described below.
R¹ is —NHR¹⁸;
R² is H, alkyl or R² and either R⁴ or R⁵ together with the atoms to which they are attached form a cyclic alkyl group, or R² and R⁶ together with the atoms to which they are attached form a cyclic alkyl group, an aryl group or a heteroaryl group, or R² and R⁸ together with the atoms to which they are attached form a cyclic heteroalkyl group;
R³ is H, alkyl or R³ and R¹⁰ together with the atoms to which they are attached form a cyclic alkyl group;
R⁴ is H, alkyl or R⁴ and R⁶ together with the atoms to which they are attached form a cyclic alkyl group, or R⁴ and R⁸ together with the atoms to which they are attached form a cyclic heteroalkyl group, or R⁴ and R¹⁰ together with the atoms to which they are attached form a cyclic alkyl group;
R⁵ is H, F, Cl , OR¹⁶, SR¹⁶ or S(O)ₙR¹⁷, or R⁵ and R⁶ together with the atoms to which they are attached form a cyclic alkyl group, or R⁵ and R⁸ together with the atoms to which they are attached form a cyclic heteroalkyl group;
R⁶ is H, alkyl or R⁶ and R² together with the atoms to which they are attached form a cyclic alkyl group, an aryl group or a heteroaryl group, or R⁶ and R⁴ together with the atoms to which they are attached form a cyclic alkyl group, or R⁶ and R⁵ together with the atoms to which they are attached form a cyclic alkyl group;
R⁷ is H, alkyl or R⁷ and R¹⁰ together with the atoms to which they are attached form a cyclic alkyl group;
R⁸ is H, alkyl, or R⁸ and R² together with the atoms to which they are attached form a cyclic heteroalkyl group, or R⁸ and R⁴ together with the atoms to which they are attached form a cyclic heteroalkyl group, or R⁸ and R⁵ together with the atoms to which they are attached form a cyclic heteroalkyl group, or R⁸ and R⁶ together with the atoms to which they are attached form a cyclic heteroalkyl group, or R⁸ and R¹¹ together with the atoms to which they are attached form a cyclic heteroalkyl group;
R⁹ is H or alkyl;
R¹⁰ is H or alkyl, or R¹⁰ and R³ together with the atoms to which they are attached form a cyclic alkyl group, or R¹⁰ and R⁴ together with the atoms to which they are attached form a cyclic alkyl group, or R¹⁰ and R⁷ together with the atoms to which they are attached form a cyclic alkyl group, or R¹⁰ and R¹¹ together with the atoms to which they are attached form a cyclic alkyl group;
R¹¹ is H, alkyl, OH, SH or F, or R¹¹ and R⁸ together with the atoms to which they are attached form a cyclic heteroalkyl group, or R¹¹ and R¹⁰ together with the atoms to which they are attached form a cyclic alkyl group;
R¹² is H or alkyl;
R¹³ is H or alkyl when X is N;
R¹³ is not a substituent when X is O;
R¹⁴ is —CHR¹⁵—CO₂H, —C₆H₄—CO₂H, —C₄H₃N—CO₂H, —C₄H₂S—CO₂H, C₄H₂O—CO₂H, —CH R¹⁵—S(O)₂OH, —CH R¹⁵—S(O)₂—NH₂ or —CH R¹⁵—P(O)(CH₃)OH; and
R¹⁵ is H or alkyl;
R¹⁶ is H or alkyl;
R¹⁷ is alkyl or aryl;
R¹⁸ is H, CH₃ or CH₂CH₃; and,
n is 1 or 2;
with the proviso that the compound of formula (I) is neither negamycin nor deoxynegamycin.

2. The compound according to claim 1, wherein R¹⁴ is —CHR¹⁵—CO₂H.

3. The compound according to claim 1, wherein R⁵ is OH.

4. The compound according to claim 1, wherein R⁵ is F.

5. The compound according to claim 1, wherein R² and R⁶ together with the atoms to which they are attached form a cyclic alkyl group, an aryl group or a heteroaryl group.

6. The compound according to claim 1, wherein R² and R⁸ together with the atoms to which they are attached form a cyclic heteroalkyl group.

7. The compound according to claim 1, wherein either R⁴ or R⁵ and R⁶ together with the atoms to which they are attached form a cyclic alkyl group.

8. The compound according to claim 1, wherein either R⁴ or R⁵ and R⁸ together with the atoms to which they are attached form a cyclic heteroalkyl group.

9. The compound according to claim 4, wherein R¹ is —NHR¹⁸, and wherein X is N, and wherein R², R³, R⁴, R⁶, R⁷, R⁸, R⁹, R¹⁰, R¹¹ and R¹² are H.

10. The compound according to claim 5, wherein R² and R⁶ together with the atoms to which they are attached form a cyclic alkyl group, and wherein the cyclic alkyl group is selected from a group consisting of the following cyclic alkyl groups: cyclopropyl, cyclobutyl, cyclopentyl and cyclohexyl.

11. The compound according to claim 9, wherein R¹³ is methyl and R¹⁴ is —CHR¹⁵—CO₂H.

12. The compound according to claim 10, wherein the cyclic alkyl group is cyclopropyl.

13. The compound according to claim 11, wherein R¹⁵ is H.

14. A compound selected from a group consisting of the following compounds:

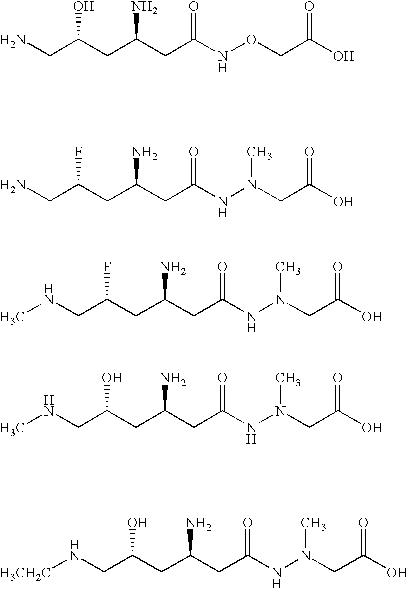

15. A method of treating a bacterial infection in a mammal, wherein the method comprises administering a compound of formula (I) to the mammal: 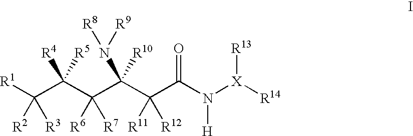
wherein,
R¹ is —NHR¹⁸;
R² is H, alkyl or R² and either R⁴ or R⁵ together with the atoms to which they are attached form a cyclic alkyl group, or R² and R⁶ together with the atoms to which they are attached form a cyclic alkyl group, an aryl group or a heteroaryl group, or R² and R⁸ together with the atoms to which they are attached form a cyclic heteroalkyl group;
R³ is H, alkyl or R³ and R¹⁰ together with the atoms to which they are attached form a cyclic alkyl group;
R⁴ is H, alkyl or R⁴ and R⁶ together with the atoms to which they are attached form a cyclic alkyl group, or R⁴ and R⁸ together with the atoms to which they are attached form a cyclic heteroalkyl group, or R⁴ and R¹⁰ together with the atoms to which they are attached form a cyclic alkyl group;
R⁵ is H, F, Cl , OR¹⁶, SR¹⁶ or S(O)ₙR¹⁷, or R⁵ and R⁶ together with the atoms to which they are attached form a cyclic alkyl group, or R⁵ and R⁸ together with the atoms to which they are attached form a cyclic heteroalkyl group;
R⁶ is H, alkyl or R⁶ and R² together with the atoms to which they are attached form a cyclic alkyl group, an aryl group or a heteroaryl group, or R⁶ and R⁴ together with the atoms to which they are attached form a cyclic alkyl group, or R⁶ and R⁵ together with the atoms to which they are attached form a cyclic alkyl group;
R⁷ is H, alkyl or R⁷ and R¹⁰ together with the atoms to which they are attached form a cyclic alkyl group;
R⁸ is H, alkyl, or R⁸ and R² together with the atoms to which they are attached form a cyclic heteroalkyl group, or R⁸ and R⁴ together with the atoms to which they are attached form a cyclic heteroalkyl group, or R⁸ and R⁵ together with the atoms to which they are attached form a cyclic heteroalkyl group, or R⁸ and R⁶ together with the atoms to which they are attached form a cyclic heteroalkyl group, or R⁸ and R¹¹ together with the atoms to which they are attached form a cyclic heteroalkyl group;
R⁹ is H or alkyl;
R¹⁰ is H or alkyl, or R¹⁰ and R³ together with the atoms to which they are attached form a cyclic alkyl group, or R¹⁰ and R⁴ together with the atoms to which they are attached form a cyclic alkyl group, or R¹⁰ and R⁷ together with the atoms to which they are attached form a cyclic alkyl group, or R¹⁰ and R¹¹ together with the atoms to which they are attached form a cyclic alkyl group;
R¹¹ is H, alkyl, OH, SH or F, or R¹¹ and R⁸ together with the atoms to which they are attached form a cyclic heteroalkyl group, or R¹¹ and R¹⁰ together with the atoms to which they are attached form a cyclic alkyl group;
R¹² is H or alkyl;
R¹³ is H or alkyl when X is N;
R¹³ is not a substituent when X is O;
R¹⁴ is —CHR¹⁵—CO₂H, —C₆H₄—CO₂H, —C₄H₃N—CO₂H, —C₄H₂S—CO₂H, C₄H₂O—CO₂H, —CH R¹⁵—S(O)₂OH, —CH R¹⁵—S(O)₂—NH₂ or —CH R¹⁵—P(O)(CH₃)OH; and
R¹⁵ is H or alkyl;
R¹⁶ is H or alkyl;
R¹⁷ is alkyl or aryl;
R¹⁸ is H, CH₃ or CH₂CH₃; and,
n is 1 or 2;
with the proviso that the compound of formula (I) is neither negamycin nor deoxynegamycin.

16. The method according to claim 15, wherein the method comprises administration of a compound of formula (I) in conjunction with another antibiotic.

17. The method according to claim 15, wherein R⁵ is F.

18. The method according to claim 15, wherein R² and R⁶ together with the atoms to which they are attached form a cyclic alkyl group, and wherein the cyclic alkyl group is selected from a group consisting of the following cyclic alkyl groups: cyclopropyl, cyclobutyl, cyclopentyl and cyclohexyl.

19. The method according to claim 16, wherein the other antibiotic is a gram positive agent.

20. The method according to claim 17, wherein R¹ is —NHR¹⁸, and wherein X is N, and wherein R², R³, R⁴, R⁶, R⁷, R⁸, R⁹, R¹⁰, R¹¹ and R¹² are H.

21. The method according to claim 18, wherein the cyclic alkyl group is cyclopropyl.

22. The method according to claim 18, wherein R¹³ is methyl, and wherein R¹⁴ is —CHR¹⁵—CO₂H, and wherein R¹⁵ is H.

23. A method of treating a bacterial infection in a mammal, wherein the method comprises administering a compound to the mammal, and wherein the compound is selected from a group consisting of the following compounds:

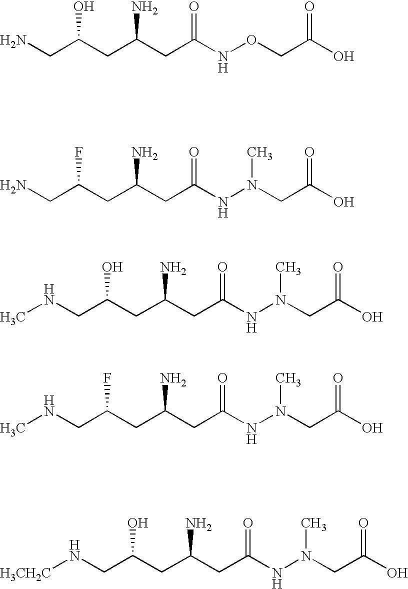

24. A pharmaceutical composition, wherein the composition comprises a pharmaceutically acceptable carrier and a therapeutically effective amount of a compound of formula (I):
wherein, 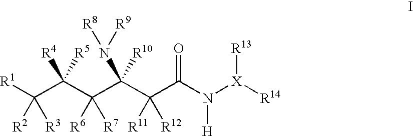
R¹ is —NHR¹⁸;
R² is H, alkyl or R² and either R⁴ or R⁵ together with the atoms to which they are attached form a cyclic alkyl group, or R² and R⁶ together with the atoms to which they are attached form a cyclic alkyl group, an aryl group or a heteroaryl group, or R² and R⁸ together with the atoms to which they are attached form a cyclic heteroalkyl group;
R³ is H, alkyl or R³ and R¹⁰ together with the atoms to which they are attached form a cyclic alkyl group;
R⁴ is H, alkyl or R⁴ and R⁶ together with the atoms to which they are attached form a cyclic alkyl group, or R⁴ and R⁸ together with the atoms to which they are attached form a cyclic heteroalkyl group, or R⁴ and R¹⁰ together with the atoms to which they are attached form a cyclic alkyl group;
R⁵ is H, F, Cl , OR¹⁶, SR¹⁶ or S(O)ₙR¹⁷, or R⁵ and R⁶ together with the atoms to which they are attached form a cyclic alkyl group, or R⁵ and R⁸ together with the atoms to which they are attached form a cyclic heteroalkyl group;
R⁶ is H, alkyl or R⁶ and R² together with the atoms to which they are attached form a cyclic alkyl group, an aryl group or a heteroaryl group, or R⁶ and R⁴ together with the atoms to which they are attached form a cyclic alkyl group, or R⁶ and R⁵ together with the atoms to which they are attached form a cyclic alkyl group;
R⁷ is H, alkyl or R⁷ and R¹⁰ together with the atoms to which they are attached form a cyclic alkyl group;
R⁸ is H, alkyl, or R⁸ and R² together with the atoms to which they are attached form a cyclic heteroalkyl group, or R⁸ and R⁴ together with the atoms to which they are attached form a cyclic heteroalkyl group, or R⁸ and R⁵ together with the atoms to which they are attached form a cyclic heteroalkyl group, or R⁸ and R⁶ together with the atoms to which they are attached form a cyclic heteroalkyl group, or R⁸ and R¹¹ together with the atoms to which they are attached form a cyclic heteroalkyl group;
R⁹ is H or alkyl;
R¹⁰ is H or alkyl, or R¹⁰ and R³ together with the atoms to which they are attached form a cyclic alkyl group, or R¹⁰ and R⁴ together with the atoms to which they are attached form a cyclic alkyl group, or R¹⁰ and R⁷ together with the atoms to which they are attached form a cyclic alkyl group, or R¹⁰ and R¹¹ together with the atoms to which they are attached form a cyclic alkyl group;
R¹¹ is H, alkyl, OH, SH or F, or R¹¹ and R⁸ together with the atoms to which they are attached form a cyclic heteroalkyl group, or R¹¹ and R¹⁰ together with the atoms to which they are attached form a cyclic alkyl group;
R¹² is H or alkyl;
R¹³ is H or alkyl when X is N;
R¹³ is not a substituent when X is O;
R¹⁴ is —CHR¹⁵—CO₂H, —C₆H₄—CO₂H, —C₄H₃N—CO₂H, —C₄H₂S—CO₂H, C₄H₂O—CO₂H, —CH R¹⁵—S(O)₂OH, —CH R¹⁵—S(O)₂—NH₂ or —CH R¹⁵—P(O)(CH₃)OH; and
R¹⁵ is H or alkyl;
R¹⁶ is H or alkyl;
R¹⁷ is alkyl or aryl;
R¹⁸ is H, CH₃ or CH₂CH₃; and,
n is 1 or 2;
with the proviso that the compound of formula (I) is neither negamycin nor deoxynegamycin.

25. The composition according to claim 24, wherein R⁵ is F.

26. The composition according to claim 24, wherein R² and R⁶ together with the atoms to which they are attached form a cyclic alkyl group, and wherein the cyclic alkyl group is selected from a group consisting of the following cyclic alkyl groups: cyclopropyl, cyclobutyl, cyclopentyl and cyclohexyl.

27. The composition according to claim 25, wherein R¹ is —NHR¹⁸, and wherein X is N, and wherein R², R³, R⁴, R⁶, R⁷, R⁸, R⁹, R¹⁰, R¹¹ and R¹² are H.

28. The composition according to claim 26, wherein the cyclic alkyl group is cyclopropyl.

29. The composition according to claim 27, wherein R¹³ is methyl, and wherein R¹⁴ is —CHR¹⁵—CO₂H, and wherein R¹⁵ is H.

30. A pharmaceutical composition, wherein the composition comprises a pharmaceutically acceptable carrier and a therapeutically effective amount of a compound, and wherein the compound is selected from the group consisting of the following compounds: [chemical structure]
